(19) United States
(12) Patent Application Publication
(10) Pub. No.: US 2024/0066074 A1
(43) Pub. Date: Feb. 29, 2024

(54) PROBIOTIC TREATMENTS FOR PARKINSON'S DISEASE

(71) Applicant: California Institute of Technology, Pasadena, CA (US)

(72) Inventors: Anastasiya O. MOISEYENKO, Pasadena, CA (US); Timothy R. SAMPSON, Pasadena, CA (US); Sarkis K. MAZMANIAN, Pasadena, CA (US)

(21) Appl. No.: 18/024,477

(22) PCT Filed: Sep. 7, 2021
(86) PCT No.: PCT/US21/49270

§ 371 Date: Mar. 2, 2023

Related U.S. Application Data
(60) Provisional application No. 63/077,176, filed on Sep. 11, 2020.

(51) Int. Cl. A61K 35/741; A61K 45/06; A61P 25/16

(52) U.S. Cl. CPC A61K 35/741; A61K 45/06; A61P 25/16

(57) ABSTRACT

Some embodiments herein relate generally to compositions comprising microbial organisms and/or components thereof for improving a neurological disorder, or symptoms associated with a neurological disorder, such as Parkinson's disease, and methods of using the same.

### RELATED APPLICATIONS

This application is the U.S. National Phase of International Application No. PCT/US2021/049270, filed Sep. 7, 2021, which claims the benefit of priority under 35 U.S.C. § 119(e) to U.S. Provisional App. No. 63/077,176, filed Sep. 11, 2020, each of which is hereby incorporated by reference in its entirety.

### STATEMENT REGARDING FEDERALLY SPONSORED R&D

This invention was made with government support under Grant No. NS085910 awarded by the National Institutes of Health. The government has certain rights in the invention.

### BACKGROUND

### Field

The present disclosure relates generally to probiotic compositions, which are useful for the treatment, inhibition, or amelioration of neurodegenerative diseases, such as Parkinson's disease. Embodiments include several probiotic compositions and methods of making and using these compositions.

### Description of the Related Art

Neurological dysfunction is the basis of numerous human diseases. Behavioral, psychiatric, and neurodegenerative disorders often display hallmark neuropathologies within the central nervous system (CNS). One neuropathology, amyloidosis, can result from, among other things, aberrant aggregation of specific neuronal proteins that disrupt many cellular functions. Affected tissues often contain insoluble aggregates of proteins that display altered conformations, a feature believed to contribute to an estimated 50 distinct human diseases (Sacchettini and Kelly, 2002). Neurodegenerative amyloid disorders, including Alzheimer's, Huntington's, and Parkinson's diseases (PD), are associated with various distinct amyloid proteins (Brettschneider et al., 2015). PD is the second most common neurodegenerative disease in the United States, affecting an estimated 1 million people and 1% of the US population over 60 years of age (Nails et al., 2014). Worldwide, about 3 million patients and caregivers suffer from the often-debilitating symptoms of PD, which involve motor deficits including tremors, muscle rigidity, bradykinesia, and impaired gait. It is a multifactorial disorder that has a strong environmental component, as less than 10% of cases are hereditary (Nails et al., 2014). Aggregation of α-synuclein (αSyn) is thought to be pathogenic in a family of diseases termed synucleinopathies, which includes PD, multiple system atrophy, and Lewy body dementia (Brettschneider et al., 2015; Luk et al., 2012; Prusiner et al., 2015). αSyn aggregation is a stepwise process, leading to oligomeric species and intransient fibrils that accumulate within neurons. Dopaminergic neurons of the substantia nigra pars compacta (SNpc) appear particularly vulnerable to effects of αSyn aggregates. Dopamine modulators are a first-line therapeutic in PD; however, treatments can carry serious side effects and often lose effectiveness (Jenner, 2008). Further, known treatments for these diseases target symptoms, and none address underlying disease processes. Discovery of safe and effective therapeutics are needed to address the increasing burden of PD in an ever-aging population, and to address the underlying disease processes.

### SUMMARY

Embodiments provided herein relate to compositions formulated for administration to a subject for improvement in neurodegenerative disorders, such as Parkinson's disease. Also disclosed are methods of making and using the compositions for improving, alleviating, treating, inhibiting, ameliorating, or reducing symptoms associated with neurodegenerative disorders, such as Parkinson's disease.

Some embodiments provided herein relate to compositions that include one or more isolated microbial organisms or a component of the isolated microbial organism. In some embodiments, the one or more isolated microbial organisms comprise Bacteroides, Prevotella, Faecalibacterium, a mixture thereof, or a component derived therefrom. In some embodiments, the compositions are formulated as a supplement, a powder, a pill, a tablet, a capsule, a pharmaceutical composition, a nutraceutical composition, or a probiotic composition. In some embodiments, the isolated microbial organism is cultured bacteria. In some embodiments, the components of the isolated microbial organism comprise bacterial outer membrane vesicles derived from the isolated microbial organism.

In some embodiments, the compositions consist essentially of Prevotella histicola and Faecalibacterium prausnitzii. In some embodiments, the compositions include Prevotella histicola and Faecalibacterium prausnitzii, or a component thereof. In some embodiments, the P. histicola and the F. prausnitzii are in a single composition. In some embodiments, the Prevotella histicola is present in an amount of at least 10⁸ colony forming units (cfu) and the Faecalibacterium prausnitzii is present in an amount of at least 10⁸ cfu. In some embodiments, the compositions further include Bacteroides fragilis, or a component thereof. In some embodiments, the one or more microbial organisms are whole live, lyophilized, attenuated, or killed bacteria.

In some embodiments, the pharmaceutical composition comprises a pharmaceutically acceptable carrier or excipient. In some embodiments, the nutraceutical composition is formulated as a foodstuff. In some embodiments, the foodstuff is a yogurt, kefir, fermented milk, unfermented milk, milk powder, protein powder, ice cream, smoothie, butter, spread, cream, hummus, kombucha, salad dressing, miso, tempeh, nutrition bar, snack bar, cereal, cookie, juice, or tea. In some embodiments, the probiotic composition comprises live bacteria.

In some embodiments, the compositions are formulated for oral administration. In some embodiments, the compositions are formulated in an acid-resistant formulation. In some embodiments, the compositions are formulated for controlled release within the lower intestine or colon.

In some embodiments, the compositions further include an anti-inflammatory agent. In some embodiments, the anti-inflammatory agent is aceclofenac, acetylsalicylic acid, bromfenac, celecoxib, clonixin, dexibuprofen, dexketoprofen, diclofenac, diflunisal, droxicam, etodolac, etoricoxib, fenoprofen, firocoxib, flufenamic acid, flurbiprofen, H-harpagide, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, pelubiprofen, phenylbutazone, piroxicam, rofecoxib, salicylic acid, salsalate, sulindac, tenoxicam, tolfenamic acid, tolmetin, valdecoxib, zaltoprofen, or a mixture thereof.

Some embodiments provided herein relate to any of the compositions described herein for use as a medicament. In some embodiments, the compositions for use as a medicament are for use in inhibiting, reducing, delaying, preventing, or ameliorating a neurodegenerative disorder, or one or more symptoms of a neurodegenerative disorder. In some embodiments, the neurodegenerative disorder is amyloidosis, Alzheimer's disease, Huntington's disease, Parkinson's disease, multiple system atrophy, or Lewy body dementia. In some embodiments, the one or more symptoms of a neurodegenerative disorder comprise one or more of anosmia, hyposmia, bradykinesia, ataxia, tremor, muscle rigidity, impaired gait, impaired posture and balance, loss of automatic movements, dysarthria or other speech changes, handwriting changes, orthostatic hypotension, memory deficit, dysphagia, incontinence, sleep disruption, cardiac arrhythmia, visual disturbance, psychiatric problems including depression and visual, auditory, olfactory, or tactile hallucinations, vertigo, cognitive dysfunction, altered dopamine levels, altered serotonin levels, altered kynurenine levels, and/or any combination thereof.

Some embodiments provided herein relate to methods of inhibiting, reducing, delaying, preventing, or ameliorating a synucleinopathy, or one or more symptoms of a synucleinopathy. In some embodiments, the methods include administering to a subject in need any of the compositions described herein. In some embodiments, the methods include administering to the subject in need a composition including Prevotella histicola or Faecalibacterium prausnitzii, or mixture or component thereof. In some embodiments, the methods include administering to the subject in need a composition consisting essentially of Prevotella histicola and Faecalibacterium prausnitzii, or a component thereof.

In some embodiments, the synucleinopathy is a neurodegenerative disorder, an enteric nervous system disorder, or inflammation associated thereto. In some embodiments, the synucleinopathy is amyloidosis, Alzheimer's disease, Huntington's disease, Parkinson's disease, multiple system atrophy, pure autonomic failure, or Lewy body dementia. In some embodiments, the subject is one that has been identified or selected as being at risk for developing or already having Parkinson's disease, such as by clinical or diagnostic evaluation. In some embodiments, the subject is selected as in need of the composition if a presence of a bacterial protein or a microorganism that produces the bacterial protein is detected in an intestinal sample obtained from the subject, or if a level of the bacterial protein or the microorganism that produces the bacterial protein in the intestinal sample is greater than a predetermined level or control. In some embodiments, the subject is selected as in need of the composition if a presence of Prevotella histicola or Faecalibacterium prausnilzii in an intestinal sample is lower than a predetermined level or control. In some embodiments, the composition is administered following appearance of one or more symptom or conditions of a neurodegenerative disorder. In some embodiments, the composition is administered prior to appearance of one or more symptom or conditions of a neurodegenerative disorder. In some embodiments, the one or more symptoms or conditions of a neurodegenerative disorder comprise one or more of anosmia, hyposmia, bradykinesia, ataxia, tremor, muscle rigidity, impaired gait, impaired posture and balance, loss of automatic movements, dysarthria or other speech changes, handwriting changes, orthostatic hypotension, memory deficit, dysphagia, incontinence, sleep disruption, cardiac arrhythmia, visual disturbance, psychiatric problems including depression and visual, auditory, olfactory, or tactile hallucinations, vertigo, cognitive dysfunction, altered dopamine levels, altered serotonin levels, altered kynurenine levels, and/or any combination thereof. In some embodiments, the subject in need has an abnormal level of aggregation of α-synuclein (αSyn).

In some embodiments, the methods further include identifying the subject in need thereof. In some embodiments, identifying includes measuring a rate and/or level of αSyn aggregation in the subject, measuring a clearance rate and/or level of insoluble αSyn protein aggregate in the subject, or a combination thereof. In some embodiments, the rate and/or level of αSyn aggregation in the subject, the clearance rate and/or level of insoluble αSyn protein aggregate in the subject, or a combination thereof is measured in the brain of the subject. In some embodiments, the methods further include measuring the rate and/or level of αSyn aggregation in the subject, measuring the clearance rate and/or level of insoluble αSyn protein aggregate in the subject, or a combination thereof after administering the composition to the subject.

In some embodiments, the methods improve one or more gastrointestinal functions of the subject. In some embodiments, the methods relieve constipation of the subject. In some embodiments, the methods reduce αSyn aggregated in the subject. In some embodiments, the methods reduce neuroinflammation in the subject. In some embodiments, the methods increase levels of gut Prevotella histicola or Faecalibacterium prausnitzii.

In some embodiments, the compositions are administered at least once weekly. In some embodiments, the compositions are administered at least 2-3 times weekly. In some embodiments, the compositions are administered multiple times daily.

### BRIEF DESCRIPTION OF THE DRAWINGS

FIG. 1 illustrates an exemplary method for motor function testing using P. histicola as described in some embodiments herein. Subjects were trained and baseline tested until 5 weeks of age, followed by initiation of treatment beginning at 5 weeks. Subjects were orally gavaged with vehicle or with compositions comprising P. histicola two to three times a week beginning at 5 weeks, and motor function was assessed approximately every three weeks until tissue collection at 22 weeks.

FIG. 2 graphically depicts motor function performance using various parameters over time for subjects treated beginning at 5 weeks of age with vehicle or with compositions comprising P. histicola, as outlined in FIG. 1. *p≤0.05; **p≤0.01; ***p≤0.001; ****p≤0.0001.

FIG. 3 graphically depicts levels of a. phosphorylated αSyn in midbrain; b. αSyn fibrils in midbrain; c. phosphorylated αSyn in duodenum; and d. phosphorylated αSyn in large intestine of subjects treated with vehicle or with compositions comprising P. histicola, as outlined in FIG. 1, as assayed by Western blot (a, c, and d) and dot blot (b). Subjects treated with compositions comprising P. histicola showed significantly decreased levels of phosphorylated αSyn in the midbrain and small intestine. *p≤0.05; **p≤0.01; ***p≤0.001; and ****p≤0.0001.

FIG. 4 graphically depicts graphs showing motor function performance over time of subjects treated beginning at 5 weeks of age with vehicle or with compositions comprising B. fragilis and P. histicola. *p≤0.05; **p≤0.01; ***p≤0.001; and ****p <0.0001.

FIG. 5 graphically depicts phosphorylated αSyn levels (a, b, e, f) and αSyn fibril levels (c and d) in the midbrain (a and c), striatum (b and d), duodenum (e), and proximal large intestine (f) of subjects treated with vehicle or with compositions comprising B. fragilis and P. histicola, as assayed by Western blot (a, b, e, and f) and dot blot (c and d). Subjects treated with compositions comprising B. fragilis and P. histicola showed significantly decreased levels of phosphorylated αSyn in the midbrain. *p≤0.05; **p≤0.01; ***p≤0.001; and ****p≤0.0001.

FIG. 6 graphically depicts graphs showing motor function performance over time of subjects treated beginning at 5 weeks of age with vehicle or with compositions comprising B. fragilis. Treatment with compositions comprising B. fragilis and P. histicola are more robust than treatment with compositions comprising B. fragilis alone. *p≤0.05; **p≤0.01; ***p≤0.001; and ****p≤0.0001.

FIG. 7 illustrates an exemplary method for motor function testing using F. prausnitzii as described in some embodiments herein. Subjects were trained and baseline tested until 5 weeks of age, followed by initiation of treatment beginning at 5 weeks of age. Subjects were orally gavaged with vehicle or with compositions comprising F. prausnitzii two to three times a week, and motor function was assessed at 15, 20, and 24 weeks until tissue collection at weeks.

FIG. 8 graphically depicts motor function performance using various parameters over time for subjects treated beginning at 5 weeks of age with vehicle or with compositions comprising F. prausnitzii, as outlined in FIG. 7. *p≤0.05; **p≤0.01; ***p≤0.001; ****p≤0.0001.

### DETAILED DESCRIPTION

Embodiments of the present disclosure relate to compositions, which are useful for reducing, ameliorating, inhibiting, improving, or treating a neurodegenerative disorder or one or more symptoms of the neurodegenerative disorder, and methods of making and using the compositions. The compositions may include one or more microbial organisms, such as probiotic bacteria, including, for example, probiotic bacteria that reduce inflammation, that reduce amyloid buildup or formation, or that improve symptoms associated with a neurodegenerative disorder, such as Parkinson's disease. Methods for making and using the compositions are also disclosed herein.

The following description provides context and examples but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

It will be readily understood that the aspects of the present disclosure, as generally described herein, can be arranged, substituted, combined, separated, and designed in a wide variety of different configurations, all of which are contemplated herein.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose.

### Compositions

Embodiments provided herein relate to compositions that include one or more isolated or cultured microbial organisms, or a component of the isolated microbial organism. In some embodiments, the compositions inhibit, reduce, delay, prevent, or ameliorate a neurodegenerative disorder, or one or more symptoms of a neurodegenerative disorder, such as Parkinson's disease.

As used herein, the term “composition” has its ordinary meaning as understood in light of the specification and refers to a product comprising the specified ingredients in the specified amounts, as well as any product which results, directly or indirectly, from combination of the specified ingredients in the specified amounts.

In some embodiments, the compositions include one or more isolated or cultured microbial organism. As used herein, the term “isolated” has its ordinary meaning as understood in light of the specification and refers to a component that is free from or substantially free from at least one component as found in nature. For example, in the context of an isolated microbial organism, the term isolated refers to an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored, or subsisted in non-naturally occurring environments. Specific examples of isolated microbial organisms include partially pure microbes, substantially pure microbes, and microbes cultured in a medium that is non-naturally occurring.

In some embodiments, the compositions include one or more isolated microbial organisms that are not found in nature. For example, the composition may include a combination of isolated bacteria that are not found in such isolation in nature or in the particular ratios or amounts provided in the composition. In some embodiments, the one or more microbial organisms are modified from those found in nature. Such modifications may include, for example, isolating, genetic modification, lyophilization, freeze-drying, or addition of components to the one or more microbial organisms, wherein such additions would not be found in nature e.g., emulsifiers, stabilizers, pre-biotics (such as, non-digestible oligosaccharides or fructo-oligosaccharides), preservatives, antibiotics, or antifungals. For instance, in some embodiments, the isolated bacteria described herein are formulated to include freeze-dried or lyophilized bacteria mixed with a stabilizer such a protein e.g., an albumin, and one or more sugars including, but not limited to, raffinose, soybean oligosaccharides, fructooligosaccharides, galactooligosaccharides, galactosyl lactose and palatinose, lactulose, lactitol, xylitol, sorbitol, mannitol, trehalose, glucose, sucrose, fructose, maltose, milk, milk powders, whey, whey protein concentrates, casein, casein hydrolysates, lactoferrin, lactoperoxidase, lactoglobulins, glycomacropeptides, lacto-saccharides, or lacto-lipids. Accordingly, the compositions provided herein are compositions that could not be found in nature and are not naturally occurring.

As used herein, the term “cultured” has its ordinary meaning as understood in light of the specification and refers to a microbial organism that is grown in an artificial medium and under artificial conditions to control the growth, quantity, purity, isolation, and components of the microbial organism, such as for formulation in a composition as described herein.

As used herein, the term “microbial organism” has its ordinary meaning as understood in light of the specification and refers to any organism that exists as a microscopic cell that is included within the domains of archaea, bacteria, or eukarya. Therefore, the term is intended to encompass prokaryotic or eukaryotic cells or organisms having a microscopic size and includes bacteria, archaea, and eubacteria of all species as well as eukaryotic microorganisms such as yeast and fungi. The term also includes cell cultures of any species that can be cultured for formulation in the compositions described herein.

In some embodiments, the one or more isolated microbial organisms are whole live bacteria. In some embodiments, the one or more isolated microbial organisms are attenuated bacteria. In some embodiments, the one or more isolated microbial organisms are killed bacteria. In some embodiments, the one or more isolated microbial organisms are lyophilized bacteria. In some embodiments, the one or more isolated microbial organisms are mixed with a preservative, antibiotic, antifungal, pre-biotic or stabilizer such as a protein e.g., an albumin, and one or more sugars including, but not limited to, raffinose, soybean oligosaccharides, fructooligosaccharides, galactooligosaccharides, galactosyl lactose and palatinose, lactulose, lactitol, xylitol, sorbitol, mannitol, trehalose, glucose, sucrose, fructose, maltose, milk, milk powders, whey, whey protein concentrates, casein, casein hydrolysates, lactoferrin, lactoperoxidase, lactoglobulins, glycomacropeptides, lacto-saccharides, or lacto-lipids.

In some embodiments, the one or more microbial organisms are capable of reducing or inhibiting inflammation in a subject, reducing amyloid buildup or formation in a subject, or improving symptoms associated with a neurodegenerative disorder, such as Parkinson's disease in a subject.

In some embodiments, the one or more microbial organisms included in any one or more of the compositions described herein are a bacterium from Bacteroides, Prevotella, or Faecalibacterium. Bacteroides includes Gram-negative, anaerobic, bacteria, including, for example, B. fragilis, B. thetaiotaomicron, B. nordii, B. eggerthii, or B. vulgatus. Prevotella includes Gram-negative bacteria included in the gut microbiota, including, for example, P. histicola, P. albensis, P. multiformis, P. intermedia, P. nigrescens, P. pallens, P. denticola, or P. veroralis. Faecalibacterium includes gram-positive, mesophilic, rod-shaped, anaerobic bacteria, including, for example, F. prausnitzii, an abundant commensal bacterium of the human gut microbiota. In some embodiments, the composition includes P. histicola or F. prausnitzii. In some embodiments, the composition includes P. histicola and F. prausnitzii. In some embodiments, the composition consists of P. histicola or F. prausnitzii. In some embodiments, the composition consists of P. histicola and F. prausnitzii. In some embodiments, the composition consists essentially of P. histicola or F. prausnitzii. In some embodiments, the composition consists essentially of P. histicola and F. prausnitzii. In some embodiments, the composition includes P. histicola or F. prausnitzii and B. fragilis. In some embodiments, the composition includes P. histicola, F. prausnitzii and B. fragilis. In some embodiments, the composition consists of or consists essentially of P. histicola, F. prausnitzii and B. fragilis.

Prevotella histicola is a gram-negative, obligately anaerobic, commensal microbe. P. histicola produces short chain fatty acids such as acetic acid and isovaleric acid as end products (Downes et al., 2008) and has been shown to have an anti-inflammatory effect (Marietta et al., 2016, Mangalam et al., 2017). Faecalibacterium prausnitzii is gram-positive, strictly anaerobic, and is one of the most abundant human commensal microbes (Martin et al., 2017). F. prausnitzii is a major producer of butyrate in the gut (Duncan, 2002) and has also been shown to have anti-inflammatory effects (Sokol et al., 2008, Sarrabayrouse et al., 2014). Growing evidence shows that Parkinson's disease (PD) may be linked with or even originate in the gut (Braak et al., 2003, Mulak & Bonaz, 2015, Liddle 2018). In several comparison studies of PD vs. healthy patient microbiomes, Prevotella and Faecalibacterium sp. were consistently shown to be decreased in the PD microbiome (Scheperjans et al., 2015, Keshavarzian et al., 2015, Petrov et al., 2017). Here, we show that consistent oral gavage with P. histicola or F. prausnitzii decreases the severity of motor dysfunction, and P. histicola decreases α-synuclein inclusions in the α-synuclein overexpressing (ASO) mouse model of PD.

Without being limited by theory, P. histicola and F. prausnitzii are naturally occurring microbes in the human gut. The overall decrease of Prevotella and Faecalibacterium sp. in PD microbiome suggests that restoration of Prevotella or Faecalibacterium levels may be therapeutic. As described in some embodiments herein, consistent oral administration of compositions provided herein, including compositions that include P. histicola or F. prausnitzii ameliorates symptoms associated with neurodegenerative disorders, such as motor function. In some embodiments, administration of the compositions provided herein, including compositions that include P. histicola or F. prausnitzii, decreases disease-causing α-synuclein inclusions in a mouse model of PD.

In some embodiments, any one or more of the aforementioned microbial organisms are present in a composition in an amount at least 10⁴ colony forming units (cfu), for example at least 10⁴, 10⁵, 10⁶ , 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹² cfu, 10⁵-10⁸ cfu, 10⁵-10⁹ cfu, 10⁵-10¹⁰ cfu, 10⁵-10¹¹ cfu, 10⁵-1010¹² cfu, 10⁵-10¹² cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹² cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹² cfu, −10⁹ cfu, 10⁸-10¹⁰ cfu, 10⁸-10¹¹ cfu, 1010⁸-10¹² cfu, or 10⁸-10¹² cfu. In some embodiments, the composition, use, and/or method comprises a log phase (at 37° C.) of bacteria for administration to the subject. In some embodiments, the composition, product combination, use, and/or method comprises a stationary phase (at 37° C.) of bacteria for administration to the subject. In some embodiments, the bacteria of the composition, product combination, use, and/or method are isolated bacteria.

In some embodiments, the compositions include a component of the isolated microbial organism. Such components may include, for example, bacterial outer membrane vesicles, bacterial capsules, bacterial cell wall, bacterial cell envelope, or other components of bacteria.

Some embodiments provided herein relate to a composition that includes P. histicola and F. prausnitzii. In some embodiments, the ratio of the amount of P. histicola to F. prausnilzii in the composition is 1:1, 1:2, 1:3, 1;4, 1:5, 1:6, 1:7, 1:8, 1:9, 1:10, 1:11, 1:12, 1:13, 1:14, 1:15, 1:16, 1:17, 1:18, 1:19, 1:20 or within a range defined by any two of the aforementioned ratios. In some embodiments, the ratio of the amount of F. prausnilzii to P. histicola in the composition is 1:1, 1:2, 1:3, 1;4, 1:5, 1:6, 1:7, 1:8, 1:9, 1:10, 1:11, 1:12, 1:13, 1:14, 1:15, 1:16, 1:17, 1:18, 1:19, 1:20 or within a range defined by any two of the aforementioned ratios.

### Formulations

In some embodiments, the compositions described herein are formulated for oral administration. An oral formulation may include, for example, a pharmaceutical composition, a nutraceutical composition, or a probiotic composition. In some embodiments, the composition is formulated as a solid formulation for oral ingestion, such as a supplement, tablet, enteric coated tablet, porous matrix tablet, porous microcapsule, capsule, hard capsule, soft capsule, pill, powder, sachet, granule, chewable, gel cap, or gummy. In some embodiments, the composition is formulated as a liquid formulation for oral ingestion, such as a liquid, syrup, spray, gel, slurry, or suspension. In some embodiments, the formulations include controlled release formulations, sustained release formulations, or slow-release formulations. In some embodiments, the composition is formulated as an acid-resistant formulation. An acid-resistant formulation is one which is capable of or partially capable of withstanding dissolution by acids produced in the gut. In some embodiments, the composition is formulated for controlled release within the lower intestine or colon.

In some embodiments, the compositions are formulated into an easily ingestible supplement. In some embodiments, the pharmaceutical composition for oral administration described herein comprises an additional component that enables efficient delivery of the bacteria to the colon. In some embodiments, pharmaceutical preparation that enables the delivery of the bacteria to the colon can be used. Examples of such formulations include pH sensitive compositions, such as buffered sachet formulations or enteric polymers that release their contents when the pH becomes alkaline after the enteric polymers pass through the stomach. When a pH sensitive composition is used for formulating the pharmaceutical preparation, the pH sensitive composition can be a polymer whose pH threshold of the decomposition of the composition is between about 6.8 and about 7.5.

Another embodiment of a pharmaceutical composition useful for delivery of the bacteria to the colon is one that ensures the delivery to the colon by delaying the release of the bacteria by approximately 3 to 5 hours, which corresponds to the small intestinal transit time. In some embodiments, the pharmaceutical composition for delayed release includes a hydrogel shell. The hydrogel is hydrated and swells upon contact with gastrointestinal fluid, with the result that the contents are effectively released (released predominantly in the colon). Delayed release dosage units include bacteria-containing compositions having a material which coats or selectively coats the bacteria. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

In some embodiments, the compositions are formulated as a live biotherapeutic product (LBP). As used herein, the term “live biotherapeutic product” or “LBP” has its ordinary meaning as understood in light of the specification and refers to a product that includes live bacteria and is efficacious in the prevention, treatment, or cure of a disease or condition, and is not a vaccine.

In some embodiments, the compositions are formulated as non-pharmaceutical grade probiotics. In some embodiments, the compositions are formulated as pharmaceutical grade products. In some embodiments, the formulations are prepared by growing samples from stored stocks of bacteria in fermenters to produce large volumes of biomass. This biomass is then harvested from the fermenter and typically concentrated to remove fermentation medium and other cellular products. In some embodiments, the concentrated biomass is processed to render it processable and stable. In some embodiments in which the bacteria are to be provided in solid dosage forms, the concentrated biomass is lyophilized. In some embodiments, following lyophilization, the lyophilized biomass may optionally be mixed with excipients. In some embodiments, end products include the lyophilized biomass, for example, capsules or sachets, are formulated.

Any growth medium in which the bacteria of interest can be grown may be used. Nutrient broths may be used as a growth medium. Those skilled in the art will be familiar with growth media for use in fermenting bacteria. Examples of such growth media include those that include enzymatic digestion products of protein (referred to as peptone, tryptose and/or soytone growth media); yeast; one or more vitamins; amino acids; glycerol; and/or nutrition sources. Examples of growth media include LB Medium, LB-Miller, LB-Lennox, LB-Low Salt, M9, Terrific Broth, EnPresso B, SOB Medium, SOC Medium, 2× YT Medium, NZCYM Broth, Yeast Nitrogen Base and/or NZ Amine® Broth.

In some embodiments, the pH of the growth medium is not controlled during fermentation. The pH of the fermentation medium can be adjusted, prior to commencement of the fermentation step, for example to achieve a pH in the range of about 6 to about 8 or about 6.5 to about 7.5. While those skilled in the art will recognize that, conventionally, the pH of the fermentation medium should be carefully controlled during fermentation, in embodiments provided herein, the pH of the fermentation medium is not actively controlled (i.e. the pH is allowed to naturally vary during the fermentation without the exogenous addition of a counter acid or base to maintain a particular pH).

In some embodiments, the pH of the fermentation medium is not actively controlled for at least 10% of the lag phase. In some embodiments, the pH of the fermentation medium is not actively controlled for at least 20% of the lag phase. In some embodiments, the pH of the fermentation medium is not actively controlled for at least 30% of the lag phase. In some embodiments, the pH of the fermentation medium is not actively controlled for at least 40% of the lag phase. In some embodiments, the pH of the fermentation medium is not actively controlled for at least 50% of the lag phase. In some embodiments, the pH of the fermentation medium is not actively controlled for at least 60% of the lag phase. In some embodiments, the pH of the fermentation medium is not actively controlled for at least 70% of the lag phase. In some embodiments, the pH of the fermentation medium is not actively controlled for at least 80% of the lag phase. In some embodiments, the pH of the fermentation medium is not actively controlled for at least 90% of the lag phase. In some embodiments, the pH of the fermentation medium is not actively controlled for at least 95% of the lag phase. In some embodiments, the pH of the fermentation medium is not actively controlled for the entirety of the lag phase.

In some embodiments, the pH of the fermentation medium is not actively controlled for at least 10% of the log phase. In some embodiments, the pH of the fermentation medium is not actively controlled for at least 20% of the log phase. In some embodiments, the pH of the fermentation medium is not actively controlled for at least 30% of the log phase. In some embodiments, the pH of the fermentation medium is not actively controlled for at least 40% of the log phase. In some embodiments, the pH of the fermentation medium is not actively controlled for at least 50% of the log phase. In some embodiments, the pH of the fermentation medium is not actively controlled for at least 60% of the log phase. In some embodiments, the pH of the fermentation medium is not actively controlled for at least 70% of the log phase. In some embodiments, the pH of the fermentation medium is not actively controlled for at least 80% of the log phase. In some embodiments, the pH of the fermentation medium is not actively controlled for at least 90% of the log phase. In some embodiments, the pH of the fermentation medium is not actively controlled for at least 95% of the log phase. In some embodiments, the pH of the fermentation medium is not actively controlled for the entirety of the log phase.

In some embodiments, the pH of the fermentation medium is not actively controlled for at least 10% of the fermentation period. In some embodiments, the pH of the fermentation medium is not actively controlled for at least 20% of the fermentation period. In some embodiments, the pH of the fermentation medium is not actively controlled for at least 30% of the fermentation period. In some embodiments, the pH of the fermentation medium is not actively controlled for at least 40% of the fermentation period. In some embodiments, the pH of the fermentation medium is not actively controlled for at least 50% of the fermentation period. In some embodiments, the pH of the fermentation medium is not actively controlled for at least 60% of the fermentation period. In some embodiments, the pH of the fermentation medium is not actively controlled for at least 70% of the fermentation period. In some embodiments, the pH of the fermentation medium is not actively controlled for at least 80% of the fermentation period. In some embodiments, the pH of the fermentation medium is not actively controlled for at least 90% of the fermentation period. In some embodiments, the pH of the fermentation medium is not actively controlled for at least 95% of the fermentation period. In some embodiments, the pH of the fermentation medium is not actively controlled for the entirety of the fermentation period.

In some embodiments, the pH of the fermentation medium is not actively controlled until the pH of the fermentation medium reaches a threshold value, for example about pH 3, about pH 3.5, about pH 4, about pH 4.1, about pH 4.2, about pH 4.3, about pH 4.4, about pH 4.5, about pH 4.6, about pH 4.7, about pH 4.8, about pH 4.9 or about pH 5.

In some embodiments, the pH of the fermentation medium is not actively controlled until the pH of the fermentation medium reaches a consistent level (to one decimal point) for a period of about 2 minutes, about 3 minutes, about 5 minutes, about 7 minutes, about 10 minutes, about 15 minutes, about 20 minutes, about 25 minutes or about 30 minutes.

Irrespective of whether the pH of the fermentation medium is actively controlled during the fermentation step, a terminal pH adjustment step can be performed. In such embodiments, the terminal pH adjustment step can be performed at the end of the fermentation step, e.g. once a target yield of biomass is obtained and/or at the end of the log phase. This can be achieved by adding a pH adjusting agent to alter (for example increase) the pH by about 0.5 or more, about 1.0 or more or about 1.5 or more. In some embodiments, the pH of the mixture following the terminal pH adjustment step is about 5 to about 8, about 5.5 to about 7.5 or about 6 to about 7.

Where used, the pH adjusting agent can be basic. Suitable examples of pH adjusting agents include ammonia, sodium hydroxide, calcium hydroxide, calcium carbonate, and/or potassium hydroxide, magnesium hydroxide. Upon completion of the fermentation step, the biomass can be harvested from the fermenter for further processing. Harvesting can be achieved by removing some or all of the biomass from the fermenter via an outlet provided therein, or via any other technique known to those skilled in the art. A pumping apparatus, e.g. a peristaltic pump (e.g. operated at a rate of at least about 0.5 L per hour, at least about 1 L per hour, at least about 2 L per hour, at least about 5 L per hour or at least about 10 L per hour), can be employed to facilitate harvesting of the biomass from the fermenter. Additionally or alternatively, harvesting of the biomass can be achieved by feeding gas (e.g. an inert gas such as nitrogen) into the fermenter thus forcing out the biomass via an outlet.

Any type of fermenter can be used for growth of bacteria, provided that it is capable of housing a bacterial population and permitting and ideally promoting growth thereof. The skilled person will be familiar with such fermenters. In some embodiments, the fermenter can be a reusable fermenter, for example one formed of and/or coated with stainless steel, ceramic, plastic, or glass or can be a single use disposable fermenter, e.g. a vessel or bag formed of plastic such as polyethylene. The fermenter can be a pilot scale fermenter (e.g. one having a capacity of from about 200 mL, about 500 mL or about 1 L to about 5 L, about 10 L, about 15 L or about 20 L. In other embodiments, the fermenter can be a large-scale or commercial-scale fermenter, e.g. one having a capacity of about 100 L, about 250 L or about 500 L to about 1000 L, about 2000 L, about 3000 L, about 5000 L or about 10000 L.

The fermentation step can be conducted at a temperature of about 25° C. to about 50° C., from about 30° C. to about 45° C., or from about 35° C. to about 40° C. The temperature can be constant throughout the fermentation step or can be altered.

In some cases, the fermentation medium is agitated during the fermentation step. This can be achieved using any apparatus or techniques known to those skilled in the art, for example through use of a rotary paddle, baffles formed within the fermenter, pumping the fermentation medium around the fermenter, or the like.

The skilled person will recognize that the fermentation of bacteria typically involves a lag phase (a period following the initiation of fermentation where no substantial increase of the bacterial population happens) and a log phase (a period following the lag phase in which there is an exponential increase in the bacterial population). Techniques to determine when the lag phase ends, the log phase begins and the log phase ends, for example through the use of optical density, pH and/or viable cell count measurements may also be implemented.

In embodiments in which the fermentation medium is agitated, the degree of agitation can be altered during the fermentation step. For example, the degree of agitation can be higher during the log phase than during the lag phase. In arrangements in which agitation is achieved using a rotary paddle, the rotation speed during the log phase can be about 10 rpm or more, about 20 rpm, about 30 rpm or more, about 40 rpm or more or about 50 rpm or more greater than the rotation speed during the lag phase.

In some embodiments, the fermentation conditions are maintained until a desired yield of biomass is attained and/or until the end of the log phase. In some embodiments, this can be for a period of up to about 0.5, about 1 or about 2 to about 12, about 18 or about 24 hours. The point at which the log phase of bacterial growth in a fermenter commences and is concluded may be ascertained by methods known in the art, for example through the use of optical density, pH, and/or viable cell count measurements.

The biomass harvested from the fermenter can be in liquid form. However, in alternative embodiments, the biomass can be in solid or semi-solid form. In some embodiments in which the biomass harvested from the fermenter is liquid, the biomass can include the bacteria of interest and a supernatant. The supernatant can include growth medium, metabolites produced by the bacteria of interest and/or cellular components of the bacteria of interest. Some embodiments include the step of concentrating biomass to increase the concentration of the bacteria of interest therein. In some cases, the concentration step results in a 5× or greater, a 10× or greater, a 15× or greater or a 20× or greater concentration of the biomass. The skilled person will be familiar with techniques which can be employed to concentrate biomass. In some embodiments, concentration of the biomass can be achieved via centrifugation, e.g. at a force of at least about 1000 g, about 2000 g or about 5000 g and/or cross flow (tangential) filtration optionally using a microfiltration membrane (e.g. a membrane having pores of about 0.1 to about 1 μm) and/or an ultrafiltration membrane (e.g. a membrane having pores of about 0.001 μm to about 0.1 μm).

The concentration step can be conducted at a temperature lower than the operating temperature of the fermenter during the fermentation step, but optionally above 0° C. In some embodiments, the concentration step is conducted at a temperature of about 30° C. or lower, about 20° C. or lower or about 10° C. or lower than the operating temperature of the fermenter during the fermentation step.

In some embodiments, the compositions can be subjected to a washing step. This can be performed prior to harvesting of the biomass from the fermenter (e.g. while the biomass is still contained within the fermenter or following harvesting of the biomass from the fermenter. In embodiments in which the washing step is carried out following harvesting of the biomass from the fermenter, the washing step can be carried out prior to, during or following the step of concentrating the biomass. For example, in embodiments in which the washing step is carried out during the step of concentrating the biomass, the concentration step can be initiated (e.g. centrifugation can be started) and then paused while the washing step is carried out, before the concentration step is resumed. Alternatively, the concentration step can be operated simultaneously with the washing step.

In the washing step, the biomass may be contacted with a washing fluid and the resulting mixture optionally agitated. In some embodiments, the washing fluid includes a liquid medium, which can be an aqueous liquid medium. For example, the washing fluid can be water, saline solution (e.g. 0.9% saline solution) or a mineral solution. In some embodiments, the washing fluid is not a strict saline solution, such that it does not include only sodium and chloride ions or is not formed by dissolving only sodium chloride in an aqueous medium, but additionally includes ions from metals other than sodium and/or anions other than chloride, for example, the washing fluid is made by adding a salt other than sodium chloride to the washing fluid.

In embodiments in which a mineral solution is used as the washing fluid, the mineral solution can, in some cases, include metal ions. In such embodiments, the metal ions can include ions from Group I or Group II of the periodic table, such as sodium, potassium, magnesium, calcium, lithium and/or beryllium. Additionally or alternatively, the washing fluid includes ions from one metal, more than one metal, at least two metals, at least three metals, or at least four metals.

In embodiments in which the washing fluid is a saline solution, the washing fluid includes ions from at least one metal other than sodium and/or at least one other anion in addition to chloride.

Where the washing fluid includes metal ions, these can be present at a concentration of about 0.01 wt % or higher, about 0.02 wt % or higher, or about 0.05 wt % or higher. Additionally or alternatively, the concentration of metal ions present in the washing fluid can be about 5 wt % or lower, about 2 wt % or lower, about 1 wt % or lower, about 0.5 wt % or lower or about 0.2 wt % or lower. In some embodiments, a metal ion can be present at a concentration of from about 0.01 wt % to about 5 wt %, from about 0.01 wt % to about 4 wt %, from about 0.01 wt % to about 3 wt %, from about 0.01 wt % to about 2 wt %, from about 0.01 wt % to about 1 wt %, from about 0.01 wt % to about 0.5 wt %, or from about 0.01 wt % to about 0.1 wt %.

The metal ions present in the washing fluid can be provided by adding metal salts into an aqueous medium. Any salt can be utilized to provide a source of a desired mineral in a wash solution. Such salts can include acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecyl sulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxy ethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate. In some embodiments, sodium ions can be provided by adding one or more of sodium chloride, sodium bicarbonate, sodium sulphate, sodium fluoride, sodium bromide, sodium carbonate or sodium amide to an aqueous medium. Potassium ions can be provided by adding one or more of potassium phosphate (e.g. potassium phosphate monobasic and/or potassium phosphate dibasic), potassium chloride, potassium hydroxide, potassium carbonate or potassium nitrate to an aqueous medium. Magnesium ions can be provided by adding one or more of magnesium chloride, magnesium carbonate, magnesium citrate, magnesium hydroxide, magnesium oxide or magnesium sulphate to an aqueous medium. Calcium ions can be provided by adding one or more of calcium chloride (e.g. monohydrate and/or dihydrate), calcium carbonate, calcium phosphate, calcium citrate or calcium lactate. Lithium ions can be provided by adding one or more of lithium chloride, lithium sulphate, lithium chloride, lithium bromide or lithium iodide to an aqueous medium. Beryllium ions can be provided by adding one or more of beryllium chloride, beryllium fluoride, beryllium hydroxide, beryllium sulphate, beryllium carbonate, beryllium nitrate or beryllium phosphate to an aqueous medium.

In embodiments in which sodium ions are provided by the addition of sodium chloride, at least one, at least two, at least three or at least four additional salts (preferably those listed in the preceding paragraph) are also added to the washing fluid.

In some embodiments, the washing fluid can be a buffered solution. However, as explained above, it has been found that avoiding pH control of the biomass can provide advantages in terms of the stability of products prepared from the biomass, and thus in certain embodiments, the washing fluid is not buffered.

The washing fluid can includes other ingredients, for example an antioxidant (such as cysteine, arginine, ascorbic acid (and salts and esters thereof e.g. ascorbyl palmitate, sodium ascorbate), butylated agents such as butylated hydroxyanisole or butylated hydroxytoluene, citric acid, erythorbic acid, fumaric acid, glutamic acid, glutathione, malic acid, methionine, monothioglycerol, pentetic acid, metabisulfite (such as sodium metabisulfite, potassium metabisulfite), propionic acid, propyl ga Mate, uric acid, sodium formaldehyde sulfoxylate, sulphite (e.g. sodium sulphite), sodium thiosulfate, sulphur dioxide, thymol, tocopherol (free or esterified), uric acid (and salts thereof) and salts and/or esters thereof), pH adjusting agents (such as sulphate (e.g. ammonium sulphate) and/or one or more of the other pH adjusting agents discussed herein).

In some embodiments, the compositions described herein are formulated as pharmaceutical grade bacterial biomass.

Once the concentration step and washing step (if performed) have been completed, the concentrated biomass can then be lyophilized to produce a lyophilized composition including lyophilized biomass. Prior to lyophilization, a lyophilization buffer (lyobuffer) composition including one or more excipients can be blended (collectively or separately) with the concentrated biomass. Such excipients can include: cryo protectants (e.g. polyol such as ethylene glycol, sorbitol, propylene glycol, and/or glycerol; DMSO; skim milk; yeast extract; bovine serum albumin (BSA); starch hydrolysates; saccharides (including monosaccharides, disaccharides and/or polysaccharides) such as glucose, maltose, maltotriose, trehalose, mannitol, dextran, maltodextrin, lactose and/or sucrose; and/or amino acids such as cysteine, glutamic acid (optionally in the form of a salt, such as sodium glutamate), arginine and/or glycine), antioxidants (e.g cysteine, arginine, ascorbic acid (and salts and esters thereof e.g. ascorbyl palmitate, sodium ascorbate), butylated agents such as butylated hydroxyanisole or butylated hydroxytoluene, citric acid, erythorbic acid, fumaric acid, glutamic acid, glutathione, malic acid, methionine, monothioglycerol, pentetic acid, metabisulfite (such as sodium metabisulfite, potassium metabisulfite), propionic acid, propyl gallate, uric acid, sodium formaldehyde sulfoxylate, sulphite (e.g. sodium sulphite), sodium thiosulfate, sulphur dioxide, thymol, tocopherol (free or esterified), uric acid (and salts thereof) and salts and/or esters thereof), bulking agents (e.g. mannitol, maltodextrin and/or glycine), buffers (e.g. phosphate, citrate, tris and/or Hepes), and/or surfactants (e.g. polysorbate and/or sorbitan).

In certain embodiments, the dry weight ratio of lyobuffer composition:lyophilised biomass in the lyophilized composition is about 1.5:1 or higher, about 2:1 or higher, about 3:1 or higher or about 4:1 or higher.

The lyobuffer composition in the lyophilized composition may include any of the lyobuffer excipients provided herein. For example, the lyobuffer composition may include a cryoprotectant, an antioxidant, a bulking agent, a buffer and/or a surfactant.

In preferred embodiments, a cryoprotectant is included in the lyobuffer composition. A cryoprotectant may include a monosaccharide, a disaccharide, or a polysaccharide. In certain embodiments, the lyobuffer composition may include a monosaccharide and a disaccharide, a monosaccharide and a polysaccharide, or a disaccharide and a polysaccharide. In such embodiments, one or both of those saccharides are present at the same dry weight amount, or higher, than the dry weight of the lyophilised biomass. For example, the lyobuffer composition may include a disaccharide (e.g. sucrose) and a polysaccharide (e.g. maltodextrin) and each of the disaccharide and the polysaccharide may be present at levels (calculated on a dry weight basis) equal to or greater than the lyophilized biomass.

Additionally or alternatively, the lyobuffer composition may include an antioxidant (e.g. cysteine) and/or an amino acid (e.g. arginine, sodium glutamate and/or cysteine).

The lyophilization step can be carried out in a conventional lyophilization apparatus using known techniques. For example, the lyophilization step can be conducted in lyophilization apparatus such as pilot scale lyophilization apparatus (e.g. freeze drying apparatus having an operating shelf area of about 0.1 m² or higher, about 0.2 m² or higher, about 0.5 m² or about 2 m² or lower, about 3 m² or lower or about 4 m² or lower) or commercial scale lyophilization apparatus (e.g. freeze drying apparatus having an operating shelf area of about 5 m² or higher, about 10 m² or higher, or about 20 m² or higher, or about 50 m² or lower, about 100 m² or lower, about 150 m² or lower, or about 200 m² or lower.

Lyophilized compositions can include the bacteria described herein in an amount of at least about 1×10⁶ CFU/g, at least about 1×10⁷ CFU/g, at least about 1×10⁸ CFU/g, at least about 1×10⁹ CFU/g, or at least about 1×10¹⁰ CFU/g.

Additionally or alternatively, the loss of viable bacteria (CFU/g) of the lyophilized compositions when stored at 5° C. (±3° C.) for 3 months in moisture impermeable packaging is equal to or less than 1×10³ CFU/g, equal to or less than 1×10² CFU/g, or equal to or less than about 1×10¹ CFU/g.

Once lyophilization is complete, the lyophilized composition can be blended with one or more excipients before being provided in dosage forms. Such excipients can include diluents, stabilizers, growth stimulators, fillers, lubricants, glidants and the like.

In some embodiments, the pharmaceutical dosage form disclosed herein includes one or more pharmaceutically acceptable excipients. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, stabilizers, suspending agents, adsorbents, granulating agents, preservatives, buffers, coloring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminum silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include hydroxypropyl methylcellulose (HPMC), low substituted hydroxypropyl cellulose (L-HPC), croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulfate, mineral oil, palmitic acid, myristic acid, poloxamer, polyethylene glycol, sodium benzoate, sodium chloride, sodium lauryl sulfate, talc, zinc stearate, potassium benzoate, magnesium stearate or combinations thereof. Examples of diluents include talc, ammonium alginate, calcium carbonate, calcium lactate, calcium phosphate, calcium silicate, calcium sulfate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether b-cyclodextrin, tragacanth, trehalose, xylitol, or combinations thereof.

As used herein, a “pharmaceutical composition” has its ordinary meaning as understood in light of the specification and includes the compositions as describe herein formulated for medicinal or pharmaceutical use having acceptable pharmaceutical purity and one or more pharmaceutically acceptable excipients, carriers, or diluents that render the compositions suitable for methods of administration.

The term “physiologically acceptable” has its ordinary meaning as understood in light of the specification and defines a carrier, diluent or excipient that does not abrogate the biological activity and properties of the compound. A “pharmaceutically acceptable carrier” has its ordinary meaning as understood in light of the specification, and refers to a substance, not itself a therapeutic agent, which may facilitate the incorporation of a compound into cells or tissues. The carrier may be a liquid for the dissolution of a compound to be administered by ingestion. The carrier may be a vehicle for delivery of a therapeutic agent to a subject. The carrier may improve the stability, handling, or storage properties of a therapeutic agent. The carrier may facilitate formation of a dose unit of a composition into a discrete article such as a capsule, tablet, film coated tablet, caplet, gel cap, pill pellet, or bead, and the like suitable for oral administration to a subject.

Fillers or diluents include, but are not limited to calcium phosphate, dibasic anhydrous, calcium phosphate, dibasic dihydrate, calcium phosphate tribasic, calcium sulphate, cellulose powdered, silicified microcrystailine cellulose, cellulose acetate, compressible sugar, confectioner's sugar, dextrates, dextrin, dextrose, fructose, kaolin, lactitol, lactose, lactose monohydrate, magnesium carbonate, magnesium oxide, maltodextrin, maltose, mannitol, microcrystailine cellulose, polydextrose, simethicone, sodium alginate, sodium chloride, sorbitol, starch, pregelatinized starch, sucrose, trehalose and xylitol, or mixtures thereof.

As used herein, a “diluent” has its ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion, or inhalation. A common form of diluent in the art is a buffered aqueous solution such as, without limitation, phosphate buffered saline that is physiologically compatible with human cells and tissues.

Examples of suitable lubricants include, but are not limited to, magnesium stearate, calcium stearate, zinc stearate, stearic acid, talc, glyceryl behenate, polyethylene glycol, polyethylene oxide polymers, sodium lauryl sulfate, magnesium lauryl sulfate, sodium oleate, sodium stearyl fumarate, DL-leucine, colloidal silica, and others as known in the art. In some embodiments a lubricant is magnesium stearate.

Glidants include, but are not limited to, tribasic calcium phosphate, calcium silicate, cellulose, powdered, colloidal silicon dioxide, magnesium silicate, magnesium trisilicate, starch and talc, or mixtures thereof.

Pharmaceutically acceptable surfactants include, but are limited to both non-ionic and ionic surfactants suitable for use in pharmaceutical dosage forms. Ionic surfactants can include one or more of anionic, cationic or zwitterionic surfactants. Various useful surfactants include, but are not limited to, sodium lauryl sulfate, monooleate, monolaurate, monopalmitate, monostearate or another ester of olyoxyethylene sorbitane, sodium dioctylsulfosuccinate (DOSS), lecithin, stearyic alcohol, cetostearylic alcohol, cholesterol, polyoxyethylene ricin oil, polyoxyethylene fatty acid glycerides, and poloxamer.

Excipients can include a prebiotic. The term “prebiotic” means a non-digestible ingredient that beneficially affects the LBP by selectively stimulating the growth and/or activity of one or a limited number of bacteria. Examples of prebiotics include oligosaccharides, fructooligosaccharides and galactooligosaccharides.

As used herein, an “excipient” has its ordinary meaning as understood in light of the specification and refers to an inert substance that is added to a pharmaceutical composition to provide, without limitation, bulk, consistency, stability, binding ability, lubrication, or disintegrating ability etc., to the composition. A “diluent” is a type of excipient. In some embodiments, a pharmaceutically acceptable excipient can be selected from binders, disintegrants, surfactants, or stabilizers. Any one or more of the excipients (including binders, disintegrants, surfactants, or stabilizers) can be appropriate in the pharmaceutical composition containing the one or more microbial organism or component thereof, including in some embodiments at least one pharmaceutically acceptable carrier in accordance with the disclosure herein, provided that the one or more microbial organism or component thereof is not inactivated by the formulation and the formulation is physiologically compatible and tolerable with the specific formulation.

“Excipients” or “protectants” (including cryoprotectants and lyoprotectants) generally refer to compounds or materials that are added to ensure or increase the stability of the therapeutic agent during the spray freeze dry process and afterwards, for long term stability and flowability of the powder product. Suitable excipients are generally relatively free flowing particulate solids, do not thicken or polymerize upon contact with water, are basically innocuous when inhaled or ingested by a patient and do not significantly interact with the therapeutic agent in a manner that alters its biological activity. Suitable excipients are described below and include, but are not limited to, proteins such as human and bovine serum albumin, gelatin, immunoglobulins, carbohydrates including monosaccharides (galactose, D-mannose, sorbose, etc.), disaccharides (lactose, trehalose, sucrose, etc.), cyclodextrins, and polysaccharides (raffinose, maltodextrins, dextrans, etc.); an amino acid such as monosodium glutamate, glycine, alanine, arginine or histidine, as well as hydrophobic amino acids (tryptophan, tyrosine, leucine, phenylalanine, etc.); a methylamine such as betaine; an excipient salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics®; surfactants; and combinations thereof.

In some embodiments, the composition is formulated into a dosage form that includes the lyophilized biomass. Dosage forms including the lyophilized biomass can be prepared by punching or pressing tablet cores including the lyophilized biomass and optionally coating (e.g. enteric coating) them to provide tablets, encapsulating the lyophilized biomass into capsule shells to provide capsules, or providing the lyophilized biomass in sachets and sealing the sachets.

At least one or more binders can be used in any one or more of the formulations described herein, for example, to impart cohesive qualities to a particular formulation, thus permit the resulting dosage form to remain intact during formulation of capsules, tablets, film coated tablets, caplets, gel caps, pill pellets, or beads, suitable for administration to a subject. In some embodiments, the one or more binders are selected from microcrystalline cellulose, gelatin, sugars (including, for example, sucrose, glucose, dextrose and maltodextrin), polyethylene glycol, waxes, natural and synthetic gums, polyvinylpyrrolidone, pregelatinized starch, povidone, cellulosic polymers (including, for example, hydroxypropyl cellulose (HPC), hydroxypropyl methylcellulose (HPMC), methyl cellulose, hydroxyethyl cellulose), or hydroxypropyl cellulose (HPC), or any combinations thereof.

In some embodiments, at least one or more excipients can be used in any one or more of the formulations described herein, and such excipients can comprise one or more disintegrants. The at least one or more disintegrants can be used, for example, to facilitate disintegration of a pharmaceutical composition after administration. In some embodiments, the at least one or more disintegrants included in any one or more of the formulations described herein are selected from starches, clays, celluloses, algins, gums, or crosslinked polymers or any combinations thereof In some embodiments, the one or more disintegrants are selected from crosslinked polyvinylpyrrolidone (PVP-XL), sodium starch glycolate, alginic acid, methacrylic acid DYB, microcrystalline cellulose, crospovidone, polacriline potassium, sodium starch glycolate, starch, pregelatinized starch, or croscarmellose sodium or any combinations thereof.

In some embodiments, the at least one pharmaceutically acceptable excipient can comprise one or more surfactants. The at least one or more surfactants can be used, for example, as a wetting agent. The at least one or more surfactants can be used, for example, to improve the permeation and/or bioavailability of the compositions. In some embodiments, the at least one or more surfactants included in any one or more of the formulations described herein are selected from anionic surfactants, non-ionic surfactants, or zwitterionic surfactants or any mixture thereof. In some embodiments, the one or more surfactants are selected from poly(oxyethylene) sorbitan fatty acid ester, poly(oxyethylene) stearate, poly(oxyethylene) alkyl ether, polyglycolated glyceride, poly(oxyethylene) castor oil, sorbitan fatty acid ester, poloxamer, fatty acid salt, bile salt, alkyl sulfate, lecithin, mixed micelle of bile salt and lecithin, glucose ester vitamin E TPGS (D-α-tocopheryl polyethylene glycol 1000 succinate), or sodium lauryl sulfate or any combinations thereof.

In some embodiments, the at least one pharmaceutically acceptable excipient can comprise one or more stabilizers, cryoprotectants, or lyoprotectants. In some embodiments, the at least one or more stabilizers included in any one or more of the formulations described herein are selected from alkanizing agents, chelating agents, photoprotectants, or antioxidants or any combinations thereof.

In some embodiments, the alkanizing agent is selected from alkali metal salt additives or an alkaline earth metal salt additive or any combinations thereof. Alkali metal salt additives suitable for use in any one or more of the formulations described herein can comprise, for example, sodium carbonate, sodium hydroxide, sodium silicate, disodium hydrogen orthophosphate, sodium aluminate or other suitable alkali metal salts or any combinations thereof. Alkaline earth metal salt additives can comprise, for example, calcium carbonate, calcium hydroxide, magnesium carbonate, magnesium hydroxide, magnesium silicate, magnesium aluminate, or aluminum magnesium hydroxide or any combinations thereof.

In some embodiments, a chelating agent can be included in any one or more of the formulations described herein and exemplary chelating agents include disodium EDTA, EGTA, edetic acid, or citric acid, or any combination thereof.

In some embodiments, a photoprotectant can be used, for example, to protect the pharmaceutical composition from the chemical or physical effects of light. In some embodiments, the phytoprotectant included in any one or more of the formulations described herein is selected from titanium oxide, ferric oxide, or zinc oxide or any combination thereof.

In some embodiments, the antioxidant included in any one or more of the formulations described herein is selected from butylated hydroxyanisole (BHA), sodium ascorbate, butylated hydroxytoluene (BHT), sodium sulfite, propyl gallate, tocopherol, citric acid, malic acid, or ascorbic acid, or any mixtures thereof.

In some embodiments, the composition may be formulated to include a coating, for example, a film coating. Where film coatings are involved, coating preparations can include, for example, a film-forming polymer, or a plasticizer. Non-limiting examples of film-forming polymers suitable for use in the embodiments described herein comprise hydroxypropyl methylcellulose, hydroxypropyl cellulose, methylcellulose, polyvinyl pyrrolidine, or starches or any combinations thereof. Non-limiting examples of plasticizers suitable for use in the embodiments described herein comprise polyethylene glycol, tributyl citrate, dibutyl sebecate, or acetylated monoglyceride or any combinations thereof. Dyestuffs or pigments may be added to the pharmaceutical composition or to coatings for the pharmaceutical composition for identification or to characterize different combinations of active compound doses. For this purpose, concentrated sugar solutions may be used, which may optionally comprise gum arabic, talc, polyvinyl pyrrolidone, carbopol gel, polyethylene glycol, and/or titanium dioxide, lacquer solutions, and suitable organic solvents or solvent mixtures or any combinations thereof. Dyestuffs or pigments may be added for identification or to characterize different dosage amounts of the compositions. Non-limiting examples of dyestuffs and pigments suitable for use in the embodiments described herein comprise iron oxides of various colors, lake dyes of many colors, or titanium dioxide or any combinations thereof.

In some embodiments, the compositions may be formulated as a dietary supplement or pharmaceutical composition suitable for administration to a subject orally, rectally, transmucosally, topically, via intestinal administration, parenteral delivery (including intramuscular, subcutaneous, intravenous, and/or intramedullary injections), intrathecally, via direct intraventricular, intraperitoneal, intranasal, or intraocular injection.

In some embodiments, the compositions are formulated to be stable for at least 1 week, 2 weeks, 3 weeks, 1 month, 2 months, 3 months, 6 months, 9 months, 12 months, 15 months, 18 months, 24 months, 36 months, or 48 months or within a range defined by any two of the aforementioned times.

As used herein, the term “nutraceutical” has its ordinary meaning as understood in light of the specification and refers to a food stuff that provides health benefits. Nutraceutical foods may not be subject to the same testing and regulations as pharmaceutical drugs. Examples of nutraceutical compositions that may be ingested by a subject include dietary supplements or fortified foods.

As used herein, the term “probiotic” has its ordinary meaning as understood in light of the specification and refers to live microorganisms, which, when administered in adequate amounts, confer a health benefit on the host. A probiotic composition is one which includes the one or more microbial organism and a physiologically acceptable carrier. The probiotics may be available in foods and dietary supplements (for example, but not limited to capsules, tablets, and powders).

The probiotic composition may be a liquid formulation or a solid formulation. When the probiotic composition is a solid formulation, it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip, or a film. When the probiotic composition is a liquid formulation, it may be formulated as an oral solution, a suspension, an emulsion, or syrup. Said composition may further comprise a carrier material independently selected from, but not limited to, the group consisting of lactic acid fermented foods, fermented dairy products, resistant starch, dietary fibers, carbohydrates, proteins, and glycosylated proteins.

As used herein, the probiotic composition could be formulated as a food composition, a dietary supplement, a functional food, a medical food, or a nutritional product as long as the required effect is achieved. Said food composition may include yogurt, kefir, fermented milk, unfermented milk, milk powder, protein powder, ice cream, smoothie, butter, spread, cream, hummus, kombucha, salad dressing, miso, tempeh, nutrition bar, snack bar, health bars, cereal, cookie, juice, tea, soy beverages, breads, biscuits, crackers, gummies, and nutritional products. The food composition may further include a carrier material, such as lactic acid fermented foods, fermented dairy products, resistant starch, dietary fibers, carbohydrates, proteins, or glycosylated proteins.

In some embodiments, the compositions are formulated as a product combination. In some embodiments, the product combination includes one or more microbial organism as described herein and one or more agents for treating or preventing a neurological disorder. In some embodiments, the one or more agents for treating or preventing a neurological disorder includes an anti-inflammatory agent.

An anti-inflammatory agent is an agent that reduces redness, swelling, or pain, and may include, for example aceclofenac, acetylsalicylic acid, bromfenac, celecoxib, clonixin, dexibuprofen, dexketoprofen, diclofenac, diflunisal, droxicam, etodolac, etoricoxib, fenoprofen, firocoxib, flufenamic acid, flurbiprofen, H-harpagide, ibuprofen, indomethacin, isoxicam, ketoprofen, ketorolac, licofelone, lornoxi cam, loxoprofen, lumiracoxib, meclofenamic acid, mefenamic acid, meloxicam, nabumetone, naproxen, nimesulide, oxaprozin, parecoxib, pelubiprofen, phenylbutazone, piroxicam, rofecoxib, salicylic acid, salsalate, sulindac, tenoxicam, tolfenamic acid, tolmetin, valdecoxib, zaltoprofen, or a mixture thereof.

In some embodiments, the one or more microbial organism is administered in combination with one or more agent. In some embodiments, the composition including one or more microbial organism is coadministered with one or more agent.

As used herein, the term “coadministration” has its ordinary meaning as understood in light of the specification and refers to delivery of two or more separate chemical entities. Coadministration refers to the simultaneous delivery of separate agents; to the simultaneous delivery of a mixture of agents; as well as to the delivery of one agent followed by delivery of a second agent or additional agents. In all cases, agents that are coadministered are intended to work in conjunction with each other. Similarly, in the context of administration of more than one compound, the term “in combination” refers to a concomitant delivery of one compound with one or more compounds. The compounds may be administered in combination by simultaneous administration or administration of one compound before or after administration of another compound, such as within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes or within a range defined by any two of the aforementioned time points.

Some embodiments provided herein relate to a formulation that includes P. histicola and F. prausnitzii formulated as an orally ingestible supplement. In some embodiments, the ratio of the amount of P. histicola to F. prausnitzii in the composition is 1:1, 1:2, 1:3, 1;4, 1:5, 1:6, 1:7, 1:8, 1:9, 1:10, 1:11, 1:12, 1:13, 1:14, 1:15, 1:16, 1:17, 1:18, 1:19, 1:20 or within a range defined by any two of the aforementioned ratios. In some embodiments, the ratio of the amount of F. prausnitzii to P. histicola in the composition is 1:1, 1:2, 1:3, 1;4, 1:5, 1:6, 1:7, 1:8, 1:9, 1:10, 1:11, 1:12, 1:13, 1:14, 1:15, 1:16, 1:17, 1:18, 1:19, 1:20 or within a range defined by any two of the aforementioned ratios.

### Methods of Making Compositions

Some embodiments provided herein relate to methods of making the compositions provided herein. In some embodiments, the one or more microbial organisms are cultured using appropriate microbiological methods, such as growing the one or more microbial organisms in an anaerobic environment. Following culture, live or killed microbial organisms are isolated, or components of the microbial organisms are isolated, or any combination thereof, and are formulated into any of the formulations described herein.

Formulations may be obtained by combining the compositions described herein with one or more acceptable carriers and/or excipients, optionally grinding the resulting mixture, and processing the mixture, to obtain a pharmaceutical composition, a nutraceutical composition, or a probiotic composition that is formulated as tablets, pills, capsules, granules, dragees, a liquid, a gel, a syrup, a slurry, a spray, a suspension, a supplement, a food stuff, or other desired formulation. The formulations comprising the compositions described here may be manufactured by mixing, dissolving, granulating, dragee-making, levigating, emulsifying, encapsulating, entrapping, or tabletting processes.

### Methods of Treating Neurodegenerative Disorders

Some embodiments provided herein relate to methods of reducing, ameliorating, inhibiting, improving, or treating a neurodegenerative disorder or one or more symptoms of the neurodegenerative disorder. In some embodiments, the neurodegenerative disorder is amyloidosis, Alzheimer's disease, Huntington's disease, Parkinson's disease, multiple system atrophy, or Lewy body dementia. In some embodiments, the one or more symptoms of the neurodegenerative disorder is one or more of anosmia, hyposmia, bradykinesia, ataxia, tremor, muscle rigidity, impaired gait, impaired posture and balance, loss of automatic movements, dysarthria or other speech changes, handwriting changes, orthostatic hypotension, memory deficit, dysphagia, incontinence, sleep disruption, cardiac arrhythmia, visual disturbance, psychiatric problems including depression and visual, auditory, olfactory, or tactile hallucinations, vertigo, cognitive dysfunction, altered dopamine levels, altered serotonin levels, altered kynurenine levels, and/or any combination thereof In some embodiments, the methods include reducing inflammation, reducing amyloid buildup or formation, or improving symptoms associated with a neurodegenerative disorder.

In some embodiments, the methods include selecting a subject in need of treatment or therapy, such as a subject having a neurodegenerative disorder, suspected of having a neurodegenerative disorder, or susceptible of developing a neurodegenerative disorder. In some embodiments, the subject is selected having a disruption in gut microbiota, such as a variation in gut microbiota that result in a buildup of amyloid or synuclein levels, thereby increasing the propensity to development of a neurodegenerative disorder. Such selections can be made by the skilled person including clinicians and physicians using clinical or diagnostic evaluation. In some embodiments, the methods include administering to the subject a therapeutically effective amount of a composition comprising the one or more microbial organisms as described herein. In some embodiments, the composition is administered in combination with an agent that reduces or ameliorates a neurodegenerative disorder.

In some embodiments, the composition is administered to the subject parenterally, rectally, orally, or topically. In some embodiments, the compositions reduce inflammation, reduce amyloid buildup or formation, or improve symptoms associated with a neurodegenerative disorder.

The term “therapeutically effective amount” has its ordinary meaning as understood in light of the specification and is used to indicate an amount of an active compound, or pharmaceutical agent, that elicits the biological or medicinal response indicated. For example, a therapeutically effective amount of compound can be the amount needed to prevent, alleviate, or ameliorate symptoms of a neurological disorder or prolong the survival of the subject being administered the therapy. This response may occur in a tissue, system, animal, or human and includes alleviation of the signs or symptoms of the disorder being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication, and other factors which those skilled in the medical arts will recognize.

As used herein, the term “treat”, “treating”, or “treatment” of any disease, condition, syndrome, or disorder, or symptom thereof as described herein has its ordinary meaning as understood in light of the specification, and refers, in one embodiment, to ameliorating the disease, condition, syndrome, disorder, or symptom (i.e. slowing or arresting or reducing the development of the disease or at least one of the clinical symptoms thereof), or an underlying cause of a neurodegenerative disorder. In another embodiment, “treat”, “treating”, or “treatment” refers to alleviating or ameliorating at least one physiological or biochemical parameter associated with or causative of the disease, condition, syndrome, disorder, or symptom, including those which may not be discernible by the patient. In a further embodiment, “treat”, “treating”, or “treatment” refers to modulating the disease, condition, syndrome, disorder, or symptom. either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome, disorder, or symptom. As used herein, the terms “treating,” “treatment,” “therapeutic,” or “therapy” do not necessarily mean total cure or abolition of the disease or condition.

As used herein, the term “inhibit” refers to the reduction or prevention of a neurodegenerative disorder or a symptom of a neurodegenerative disorder, or an underlying cause of a neurodegenerative disorder. The reduction can be by 10%, 20%, 30%, 40%, 50%, 60%, 70%, 80%, 90%, or 100%, or an amount that is within a range defined by any two of the aforementioned values. As used herein, the term “delay” refers to a slowing, postponement, or deferment of an event, such as a neurodegenerative disorder or a symptom of a neurodegenerative disorder, or an underlying cause of a neurodegenerative disorder, to a time which is later than would otherwise be expected. The delay can be a delay of 10%, 20%, 30%, 40%, 50%, 60%, 70%, 80%, 90%, 100%, or an amount within a range defined by any two of the aforementioned values. The terms inhibit and delay are not to be construed as necessarily indicating a 100% inhibition or delay. A partial inhibition or delay may be realized.

As used herein, the term “neurological disorder” has its ordinary meaning as understood in light of the specification, and refers to disorders of the brain, spinal cord, cranial nerve, or autonomous nervous system that are associated with aggregation of synucleins. Such disorders may include, for example, amyloidosis, Alzheimer's disease, Huntington's disease, Parkinson's disease, multiple system atrophy, or Lewy body dementia.

The majority of cases of neurodegenerative diseases are idiopathic, which, conventionally, has made it difficult to identify the etiology of most such diseases. An emerging theory is that many neurodegenerative diseases start not in the brain or central nervous system (CNS), but in the periphery and gradually migrate to the brain over the course of many years in a slow, progressive process. Still, the molecular etiology in the periphery has been the subject of study. In the case of Parkinson's disease, it is known that constipation and hyposmia occur in many patients often decades before the emergence of the stereotypical motor symptoms that currently define Parkinson's disease. Without being limited by theory, it is therefore contemplated that α-synuclein aggregation begins in the gastrointestinal (GI) tract and in the olfactory bulb, and that aggregated α-synuclein gradually progresses to the brain in a prion-like propagative process. In this scenario, known more generally as Braak's hypothesis, it is contemplated that analysis of the molecular mechanisms involved in these peripheral tissues can lead to non-intuitive, non-conventional approaches for preventing and/or treating amyloid disorders, such as α-synucleinopathies, such as Parkinson's Disease.

As used herein, a “subject” refers to an animal that is the object of treatment, inhibition, or amelioration, observation, or experiment. “Animal” includes cold- and warm-blooded vertebrates and/or invertebrates such as fish, shellfish, or reptiles and, in particular, mammals. “Mammal” includes, without limitation, mice, rats, rabbits, guinea pigs, dogs, cats, sheep, goats, cows, horses, primates, such as monkeys, chimpanzees, and/or apes, and, in particular, humans. In some embodiments, the subject is human.

In some embodiments, the compositions that include one or more microbial organisms as disclosed herein may contain at least 10⁴ colony forming units (cfu), for example at least 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-1010¹¹ cfu, 10⁴-10¹² cfu, 10⁴-10¹² cfu, 10⁵-10⁸ cfu, 10⁵-10⁹ cfu, 10⁵-10¹⁰ cfu, 10⁵-10¹¹ cfu, 10⁵-10¹² cfu, 10⁵-10¹² cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹² cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹² cfu, −10⁹ cfu, 10⁸-10¹⁰ cfu, 10⁸-10¹¹ cfu, 10⁸-10¹² cfu, or 10⁸-10¹² cfu. In non-human animal studies, applications of potential products are commenced at higher dosage levels, with dosage being decreased until the desired effect is no longer achieved or adverse side effects disappear.

The dosage may be a single dosage or a series of two or more given in the course of one or more days, as is needed by the subject. In some alternatives, the compositions disclosed herein will be administered for a period of continuous therapy, for example for a week or more, or for months or years. In some alternatives, the compositions disclosed herein can be administered one time per day.

Multiple doses of the compositions disclosed herein can be administered to a subject. For example, the compositions disclosed herein can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or more times a day (4, 5, 6, or 7 times a day), over a period of time ranging from one day to one week, from two weeks to four weeks, from one month to two months, from two months to four months, from four months to six months, from six months to eight months, from eight months to 1 year, from 1 year to 2 years, or from 2 years to 4 years, or more.

In some alternatives, the compositions disclosed herein and an additional therapy described herein can be cyclically administered to a patient. Cycling therapy involves the administration of a first active ingredient for a period of time, followed by the administration of a second active ingredient for a period of time and repeating this sequential administration. Cycling therapy can reduce the development of resistance to one or more therapies, avoid or reduce the side effects of one or more therapies, and/or improve the efficacy of the therapy. In some alternatives, the compositions disclosed herein and an additional therapy described herein are administered in a cycle of less than 3 weeks, once every two weeks, once every 10 days, or once every week. The number of cycles can be from 1 to 12 cycles, or from 2 to 10 cycles, or from 2 to 8 cycles.

The daily dosage regimen for an adult human patient may be the same or different for two active ingredients provided in combination. In some alternatives, the active ingredient is one or more microbial organisms as described herein. In some alternatives, both an active ingredient and an additional therapy agent are administered to a subject. The dosage or each active ingredient can be, independently, a single one or a series of two or more given in the course of one or more days, as is needed by the subject. In some alternatives, the active ingredients will be administered for a period of continuous therapy, for example for a week or more, or for months or years. In some alternatives, the compositions disclosed herein can be administered several times per day. In some alternatives, the additional therapy agent can be administered once a week.

As will be understood by those of skill in the art, in certain situations it may be necessary to administer the active ingredients disclosed herein in amounts that exceed, or even far exceed, the above-stated, preferred dosage range in order to effectively and aggressively treat particularly aggressive stages of neurodegenerative disorders, or to prevent onset of such disorders.

For any of the methods for using the compositions described herein, a corresponding use is contemplated. Thus, use of the compositions for reducing, ameliorating, inhibiting, improving, or treating a neurodegenerative disorder or one or more symptoms of the neurodegenerative disorder are provided. In addition, the composition comprising one or more microbial organisms for use in reducing, ameliorating, inhibiting, improving, or treating a neurodegenerative disorder or one or more symptoms of the neurodegenerative disorder are also contemplated. In some embodiments, the compositions are for use as a medicament. Thus, some embodiments relate to the use of the composition in any of the methods disclosed herein. In some embodiments, the use relates to the manufacture of a medicament for the treatment of a disorder. As used herein, “medicament” is meant to be equivalent to “pharmaceutical formulation,” and both terms are used interchangeably.

Some embodiments provided herein relate to compositions that includes P. histicola and F. prausnitzii, formulated in an orally ingestible supplement, and used for reducing, ameliorating, inhibiting, improving, or treating a neurodegenerative disorder or one or more symptoms of the neurodegenerative disorder, such as PD.

### Additional Embodiments

Along with the disclosure above, in some embodiments, the following alternatives are set forth:
1. A composition comprising: one or more isolated microbial organisms or a component of the isolated microbial organism, wherein the one or more isolated microbial organisms comprise Bacteroides, Prevotella, Faecalibacterium, a mixture thereof, or a component derived therefrom, wherein the composition is formulated as a supplement, a powder, a pill, a tablet, a capsule, a pharmaceutical composition, a nutraceutical composition, or a probiotic composition.
2. The composition of alternative 1, wherein the isolated microbial organism is cultured bacteria.
3. The composition of any one of alternatives 1-2, wherein the components of the isolated microbial organism comprise bacterial outer membrane vesicles derived from the isolated microbial organism.
4. The composition of any one of alternatives 1-3, wherein the composition consists essentially of Prevotella histicola and Faecalibacterium prausnitzii.
5. The composition of any one of alternatives 1-3, wherein the composition comprises Prevotella histicola and Faecalibacterium prausnitzii, or a component thereof.
6. The composition of any one of alternatives 4-5, wherein the P. histicola and the F. prausnitzii are in a single composition.
7. The composition of any one of alternatives 4-6, wherein the Prevotella histicola is present in an amount of at least 10⁸ colony forming units (cfu) and the Faecalibacterium prausnilzii is present in an amount of at least 10⁸ cfu.
8. The composition of any one of alternatives 5-7, further comprising Bacteroides fragilis, or a component thereof.
9. The composition of any one of alternatives 1-8, wherein the one or more microbial organisms are whole live, lyophilized, attenuated, or killed bacteria.
10. The composition of any one of alternatives 1-9, wherein the pharmaceutical composition comprises a pharmaceutically acceptable carrier or excipient.
11. The composition of any one of alternatives 1-10, wherein the nutraceutical composition is formulated as a foodstuff.
12. The composition of alternative 11, wherein the foodstuff is a yogurt, kefir, fermented milk, unfermented milk, milk powder, protein powder, ice cream, smoothie, butter, spread, cream, hummus, kombucha, salad dressing, miso, tempeh, nutrition bar, snack bar, cereal, cookie, juice, or tea.
13. The composition of any one of alternatives 1-9, wherein the probiotic composition comprises live bacteria.
14. The composition of any one of alternatives 1-13, wherein the composition is formulated for oral administration.
15. The composition of any one of alternatives 1-14, wherein the composition is formulated in an acid-resistant formulation.
16. The composition of any one of alternatives 1-15, wherein the composition is formulated for controlled release within the lower intestine or colon.
17. The composition of any one of alternatives 1-16, further comprising an anti-inflammatory agent.
18. The composition of alternative 17, wherein the anti-inflammatory agent is aceclofenac, acetylsalicylic acid, bromfenac, celecoxib, clonixin, dexibuprofen, dexketoprofen, diclofenac, diflunisal, droxicam, etodolac, etoricoxib, fenoprofen, firocoxib, flufenamic acid, flurbiprofen, H-harpagide, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, pelubiprofen, phenylbutazone, piroxicam, rofecoxib, salicylic acid, salsalate, sulindac, tenoxicam, tolfenamic acid, tolmetin, valdecoxib, zaltoprofen, or a mixture thereof.
19. The composition of any one of alternatives 1-18 for use as a medicament.
20. The composition of any one of alternatives 1-18 for use in inhibiting, reducing, delaying, preventing, or ameliorating a neurodegenerative disorder, or one or more symptoms of a neurodegenerative disorder.
21. The composition for use of alternative 20, wherein the neurodegenerative disorder is amyloidosis, Alzheimer's disease, Huntington's disease, Parkinson's disease, multiple system atrophy, or Lewy body dementia.
22. The composition for use of any one of alternatives 20-21, wherein the one or more symptoms of a neurodegenerative disorder comprise one or more of anosmia, hyposmia, bradykinesia, ataxia, tremor, muscle rigidity, impaired gait, impaired posture and balance, loss of automatic movements, dysarthria or other speech changes, handwriting changes, orthostatic hypotension, memory deficit, dysphagia, incontinence, sleep disruption, cardiac arrhythmia, visual disturbance, psychiatric problems including depression and visual, auditory, olfactory, or tactile hallucinations, vertigo, cognitive dysfunction, altered dopamine levels, altered serotonin levels, altered kynurenine levels, and/or any combination thereof.
23. A method of inhibiting, reducing, delaying, preventing, or ameliorating a synucleinopathy, or one or more symptoms of a synucleinopathy, the method comprising: administering to a subject in need the composition of any one of alternatives 1-18.
24. The method of alternative 23, wherein the method comprises administering to the subject in need a composition comprising Prevotella histicola or Faecalibacterium prausnitzii, or mixture or component thereof.
25. The method of alternative 23, wherein the method comprises administering to the subject in need a composition consisting essentially of Prevotella histicola and Faecalibacterium prausnitzii, or a component thereof.
26. The method of any one of alternatives 23-25, wherein the synucleinopathy is a neurodegenerative disorder, an enteric nervous system disorder, or inflammation associated thereto.
27. The method of any one of alternatives 23-26, wherein the synucleinopathy is amyloidosis, Alzheimer's disease, Huntington's disease, Parkinson's disease, multiple system atrophy, pure autonomic failure, or Lewy body dementia.
28. The method of any one of alternatives 23-27, wherein said subject is one that has been identified or selected as being at risk for developing or already having Parkinson's disease, such as by clinical or diagnostic evaluation.
29. The method of any one of alternatives 23-28, wherein the subject is selected as in need of the composition if a presence of a bacterial protein or a microorganism that produces the bacterial protein is detected in an intestinal sample obtained from the subject, or if a level of the bacterial protein or the microorganism that produces the bacterial protein in the intestinal sample is greater than a predetermined level or control.
30. The method of any one of alternatives 23-29, wherein the subject is selected as in need of the composition if a presence of Prevotella histicola or Faecalibacterium prausnitzii in an intestinal sample is lower than a predetermined level or control.
31. The method of any one of alternatives 23-30, wherein the composition is administered following appearance of one or more symptom or conditions of a neurodegenerative disorder.
32. The method of any one of alternatives 23-30, wherein the composition is administered prior to appearance of one or more symptom or conditions of a neurodegenerative disorder.
33. The method of any one of alternatives 23-32, wherein the one or more symptoms or conditions of a neurodegenerative disorder comprise one or more of anosmia, hyposmia, bradykinesia, ataxia, tremor, muscle rigidity, impaired gait, impaired posture and balance, loss of automatic movements, dysarthria or other speech changes, handwriting changes, orthostatic hypotension, memory deficit, dysphagia, incontinence, sleep disruption, cardiac arrhythmia, visual disturbance, psychiatric problems including depression and visual, auditory, olfactory, or tactile hallucinations, vertigo, cognitive dysfunction, altered dopamine levels, altered serotonin levels, altered kynurenine levels, and/or any combination thereof.
34. The method of any one of alternatives 23-33, wherein the subject in need has an abnormal level of aggregation of α-synuclein (αSyn).
35. The method of any one of alternatives 23-34, further comprising identifying the subject in need thereof.
36. The method of alternative 35, wherein identifying comprises measuring a rate and/or level of αSyn aggregation in the subject, measuring a clearance rate and/or level of insoluble αSyn protein aggregate in the subject, or a combination thereof.
37. The method of alternative 36, wherein the rate and/or level of αSyn aggregation in the subject, the clearance rate and/or level of insoluble αSyn protein aggregate in the subject, or a combination thereof is measured in the brain of the subject.
38. The method of any one of alternatives 36-37, further comprising measuring the rate and/or level of αSyn aggregation in the subject, measuring the clearance rate and/or level of insoluble αSyn protein aggregate in the subject, or a combination thereof after administering the composition to the subject.
39. The method of any one of alternatives 23-38, wherein the method improves one or more gastrointestinal functions of the subject.
40. The method of any one of alternatives 23-39, wherein the method relieves constipation of the subject.
41. The method of any one of alternatives 23-40, wherein the method reduces αSyn aggregated in the subject.
42. The method of any one of alternatives 23-41, wherein the method reduces neuroinflammation in the subject.
43. The method of any one of alternatives 23-42, wherein the method increases levels of gut Prevotella histicola or Faecalibacterium prausnilzii.
44. The method of any one of alternatives 23-43, wherein the composition is administered at least once weekly.
45. The method of any one of alternatives 23-44, wherein the composition is administered at least 2-3 times weekly.
46. The method of any one of alternatives 23-45, wherein the composition is administered multiple times daily.

### EXAMPLES

Some aspects of the embodiments discussed above are disclosed in further detail in the following examples, which are not in any way intended to limit the scope of the present disclosure.

### Example 1

The following example demonstrates an embodiment of a method of culturing microbial organisms and methods of making the compositions and formulations disclosed herein.

P. histicola was cultured using appropriate microbiological method, including growing the bacteria in an anerobic environment at around 37° C. in peptone yeast glucose (PYG) media, trypticase soy broth, on agar plates with PYG media, or Brucella agar plates with sheep blood. F. prausnitzii was cultured using appropriate microbiological method, such as growing the bacteria in a strictly anerobic environment at around 37° C. in chopped meat with carbs (CMC), yeast casitone fatty acid (YCFA), or brain heart infusion (BHI) media, or on agar plates with YCFA or BHI media, or Brucella agar plates with sheep blood. If grown in liquid media, the bacteria were extracted by centrifugation and the pellet used to formulate the probiotic supplement. If grown on agar plates, the bacterial colonies were collected directly from the plate to be used in formulation.

Bacterial culture growth and preparation: Prevotella histicola was purchased from DSMZ (DSM 19854) and grown in DSMZ Medium #104 (modified PYG) under anaerobic conditions. 50 mL of media was inoculated with 10⁸ cfu of bacterial stock and grown for approximately 20 hours at 37° C., until optical density at 600 nm was ≥1.

Faecalibacterium prausnitzii was purchased from DSMZ (DSM 17677) and grown in CMC media under anaerobic conditions. 10 mL vials of media were inoculated with 10⁸ cfu of bacterial stock and grown for approximately 48 hours at 37° C., until optical density at 600 nm was ≥1. Animals were orally gavaged starting at 5 weeks of age, 2-3× a week with 100 μL of vehicle or bacterial culture and any excess put on a food pellet placed on the cage floor.

Live or killed whole P. histicola or F. prausnitzii bacterium or P. histicola or F. prausnitzii components, or any combination of the two for each respective bacterium, were used in the probiotic supplement. Any appropriate method to kill P. histicola or F. prausnitzii, such as heating or freeze-thaw cycles, were used. Any appropriate method to extract P. histicola or F. prausnitzii components were used. P. histicola or F. prausnitzii or its respective components were used in conjunction with any preparations required to incorporate the bacteria or its components into the capsule, pill, tablet, yogurt, milk, kefir, milk powder, protein powder, ice cream, juice, tea, kombucha, snack bar, cereal, cookie, or other oral delivery vehicle. While a dosage of at least 10⁸ cfu per intake of P. histicola or F. prausnitzii may be the most effective, any dosage of P. histicola or F. prausnitzii can be used to achieve a therapeutic effect in Parkinson's disease patients.

### Example 2

The following example demonstrates an embodiment of a method of using the compositions described herein for treating a mouse model of Parkinson's disease.

For the use of P. histicola or F. prausnitzii as a therapeutic for PD, a probiotic supplement or medication was formulated using the whole bacteria in its live or killed form, or its components (for example, outer membrane vesicles), as described in Example 1.

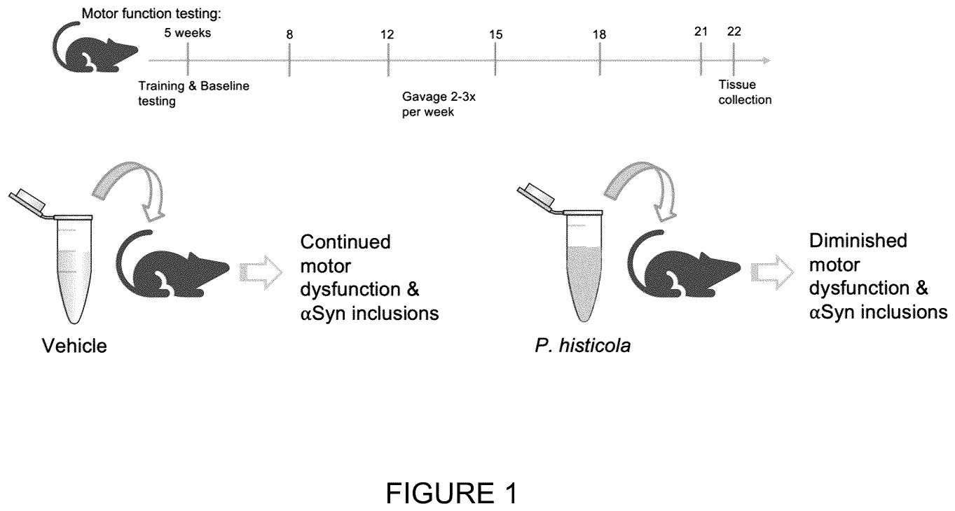

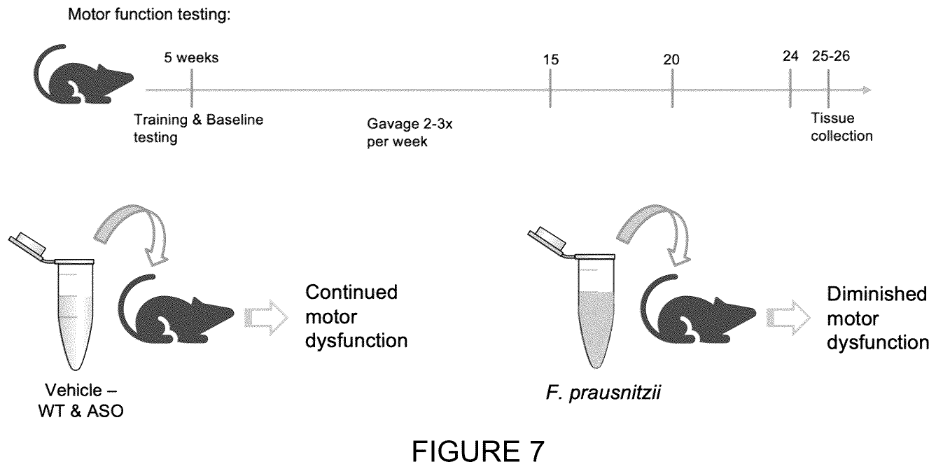

Mice: Alpha-synuclein overexpressing (ASO) male mice were bred by crossing wildtype BDF1 males with Thy1-αSyn females of the BDF1 background that were heterozygous for the Thy1 promoter-driven human alpha-synuclein transgene located on the X chromosome (Chesselet et al, 2012, Rockenstein et al., 2002). Mice had a conventional, specific pathogen free (SPF) microbiome and housed in autoclaved microisolator cages with HEPA-filtered ventilation. All behavior tests were performed between 7 and 9 hours of the light-phase, inside a biosafety cabinet in the same facility. Training and baseline testing was performed at 5 weeks of age, prior to beginning of treatment (see FIGS. 1 and 7). Treatment initiated at 5 weeks of age, 2-3 times each week, as described in Example 1. Beam traversal and pole descent training was done together over two consecutive days, with approximately an hour of rest time in between each task. On the third day, mice were tested on the beam and pole. On the subsequent day, animals were tested on wire hang and sticker removal, followed by hindlimb clasp scoring. On the next and final day, the fecal output assay was performed. Mice did not go through training again and were only tested for motor function changes about every 3 weeks up until the final time point. All data collected across trials for each task was averaged. Tissues collected were midbrain, striatum, 1 cm of duodenum, 1 cm of proximal large intestine, fecal pellets, and whole brain hemisphere.

Beam Traversal: Four 0.25 m plexiglass beam sections ranging from 3.5 cm width to 0.5 cm width, were placed together to form a 1 m beam that gradually narrows. Mice were trained for two days prior to testing day. During the first training day, mice were positioned at the beginning (thickest) section of the beam. The home cage was held sideways with its opening facing the animal, within a section's length of the beam, to encourage the animal to move forward on the beam. The animal was gently guided along the beam, until the end, where the home cage was placed sideways so that the animal was able to climb back into the cage. Three trials were performed on each training day, providing less guidance and encouragement with the home cage on each subsequent trial. On testing day, two trials were performed, with approximately 10 minute rest intervals in between trials. Timing began once the mouse crossed onto the second (2.5 cm) section of the beam and stopped once one of the forelimbs was inside the home cage. Test day trials were videotaped and later assessed in slow motion for number of steps and slips that occurred during the beam traversal. Steps made only by one limb were counted. Slips were counted if at least 3/4 of any limb left the beam.

Pole Descent: A 0.5 m pole attached to a base was used to assess the animal's ability to climb down the pole. The pole was wrapped in shelf liner to increase animals' grip. Mice were given two days of training prior to test day. Training days consisted of 3 trials. On the first training day, cage mates were removed, and the pole was placed into the home cage. The animal was placed at the bottom of the home cage briefly to show that it is in its home cage. Then, the animal was picked up by the tail and placed approximately 1/3 of the way up the pole, facing head down, and let descend. For the second trial, the animal was placed 2/3 of the way up the pole, and for the third trial, at the top of the pole. On the second day of training, the animals descended from the top of the pole for all 3 trials. On testing day, only two trials were conducted (to reduce anxiety behavior), each starting when the animal was positioned head down at the top of the pole and the tail released, ending when one of the hindlimbs touched the base of the pole.

Wire Hang: Animals were placed at the center of a 30 cm×30 cm wire mesh screen and gently flipped head over tail. A clean cage with bedding was placed 40 cm underneath the wire screen. Timing started once the animal was hanging from the wire screen horizontally and stopped when the animal fell to the bedding underneath or remained for 60 seconds. Two trials were conducted, with rest for approximately 10 minutes between trials.

Adhesive (Sticker) Removal: A circular 1/4-inch sticker was placed on the nose bridge of the animal using a tweezer. The mouse was released into the home cage, with cage mates removed, and timing was started. Timing was stopped when the mouse had successfully removed the sticker from its nose. Two trials were conducted consecutively.

Hindlimb Clasping Reflex: Mice were picked up by the tail, with the underside facing the observer, for approximately 10 seconds. Rigidity of the movement of hindlimbs was assessed on a scale of 0 to 3. A score of 0 was given if the animal was able to extend its hindlimbs outward freely, with flexibility and no clasping. A score of 1 was given if the animal had some tendency to clasp its hindlimbs inward, either with one or both, but for the majority of the restraint the limbs were still spread outward and flexible. A score of 2 was given if hindlimbs were clasped inward for the majority of time, but some movement and flexibility was still present. A score of 3 was given if the hindlimbs were clasped together entirely, with no movement or flexibility.

Fecal Output: Mice were placed into sterile semi-translucent 12 cm×25 cm plastic cups and loosely covered with a piece of sterile foil to discourage jumping out. Fecal pellets were counted every 5 minutes for 15 minutes by gently lifting the cup to see the fecal pellets collected at the bottom. After 15 minutes, mice were placed back into home cages.

Fecal Score: Following the Fecal Output assay, fecal pellets were assessed according to the Bristol stool scale. A score of 1 was given for fecal pellets that were small, dry, hard lumps, indicating severe constipation. A score of 2 was given for lumpy, dry, but sausage-like fecal pellets, indicating mild constipation. A score of 3 was given for mildly dry sausage-like pellets with cracks in the surface. A score of 4 was given for smooth, soft, sausage or snake-shaped pellets. Both scores of 3 and 4 are considered normal. A score of 5 was given for soft blobs with clear-cut edges. A score of 6 was given for fluffy pieces with ragged edges, indicating mild diarrhea. Lastly, a score of 7 was given for an entirely liquid consistency, indicating severe diarrhea. An average score was taken when animals produced fecal pellets of various scores during one fecal output session.

Western blots: Tissues were homogenized in RIPA buffer with protease inhibitor and protein levels measured using a Pierce BCA Protein Assay kit (ThermoFisher). A 4-20% SDS-PAGE was run with subsequent blotting onto a PVDF membrane. 5% skim milk in Tris-buffered saline with 0.1% Tween-20 (TBS-T) was used as a blocking buffer. Anti-pS129 α-synuclein rabbit antibody (abcam51253) was used in 1:1000 concentration with blocking buffer and incubated overnight at 4° C. Anti-IgG HRP rabbit antibody (Cell Signaling Technology) was then used in 1:1000 concentration with TB S-T and incubated for 1-2 hours at room temperature. Clarity chemiluminescence substrate (BioRad) was used for detection on a BioRad GelDoc XR and ImageJ used to perform densitometry.

Dot blots: Samples were diluted in PBS to a maximum of 1 ug of protein per 1 uL and spotted onto a nitrocellulose membrane. Anti-α-synuclein filament rabbit antibody (abcam209538) was used at a concentration of 1:1000 with blocking buffer and incubated for 1-2 hours at room temperature. All other procedure was the same as that for Western blots.

Statistical Analysis: All datasets were analyzed using GraphPad Prism 7. Two-way ANOVA was used to for comparisons between groups and two-tailed Mann-Whitney was used for pairwise comparisons.

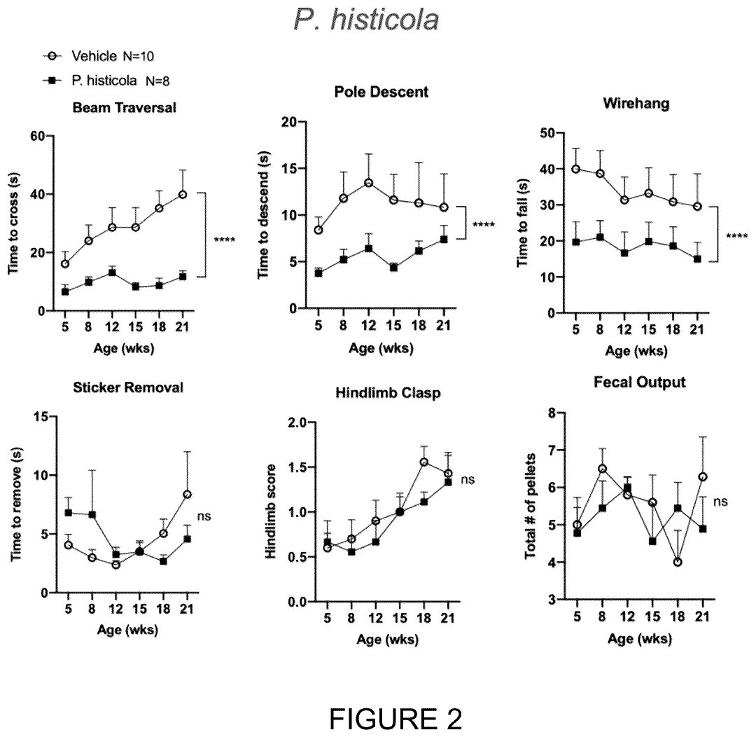

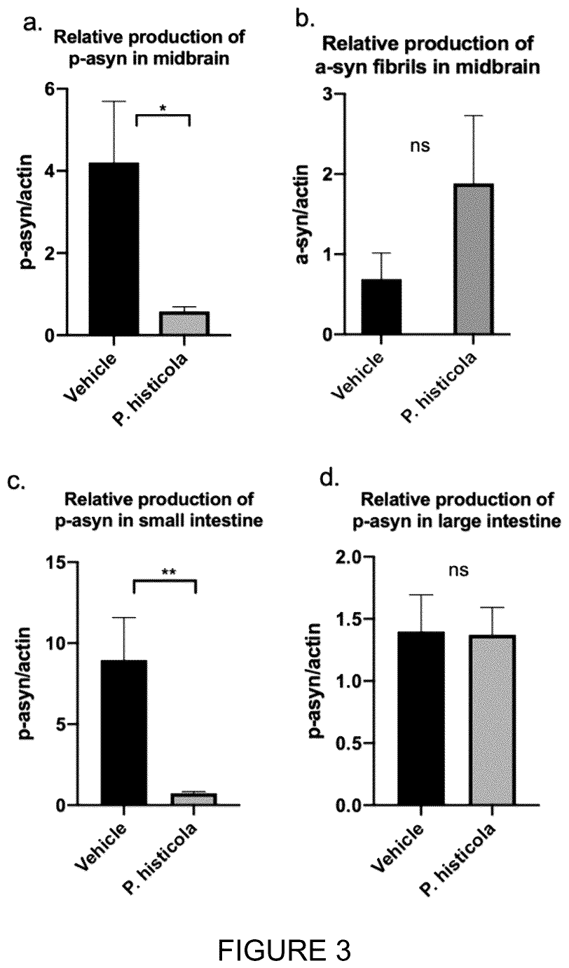

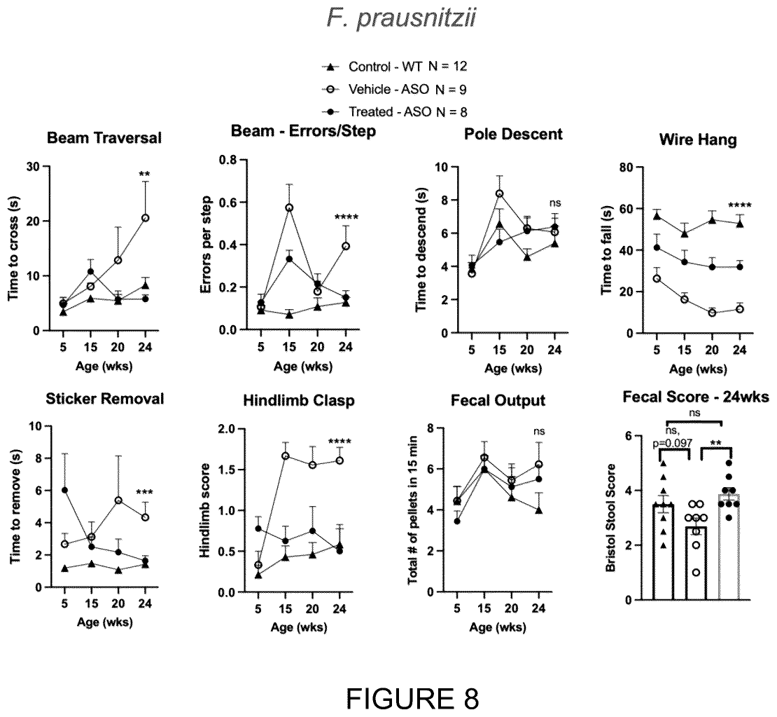

The ASO mice treated with P. histicola or F. prausnitzii exhibited increased beam traversal, pole descent, wirehang, sticker removal, hindlimp clasp, and fecal output, as compared to control mice models treated with vehicle, as shown in FIG. 2 and FIG. 8. In addition, ASO mice exhibited decreased levels of phosphorylated αSyn levels in the midbrain and small intestine compared to control, as shown in FIG. 3. Thus, ASO mice treated with the compositions described herein exhibited diminished motor dysfunction and αSyn inclusions as compared to ASO mice treated with control (see FIG. 1 and FIG. 7).

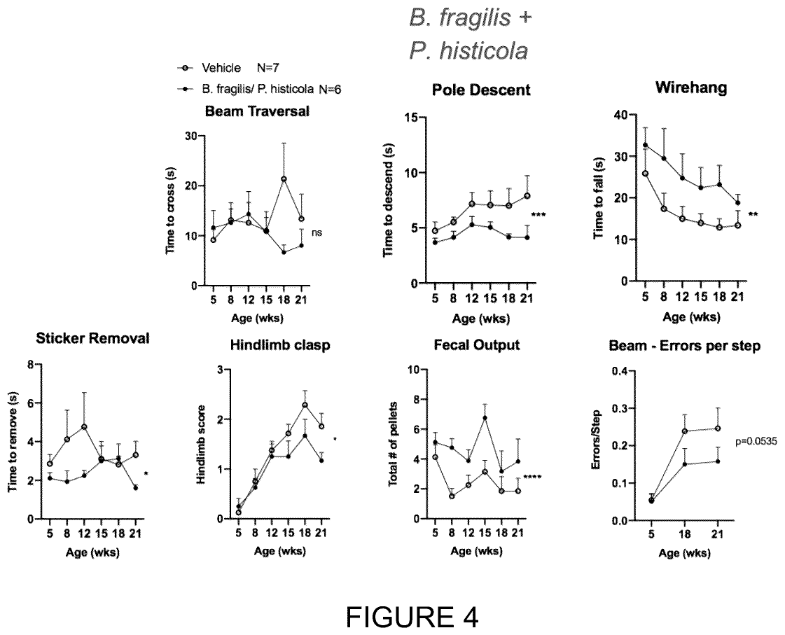

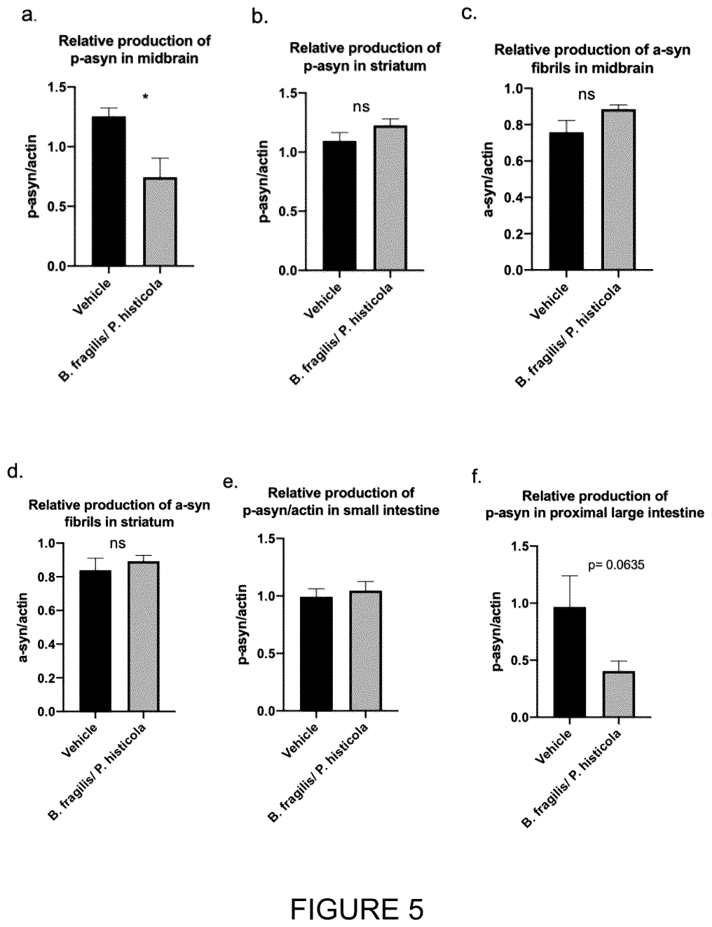

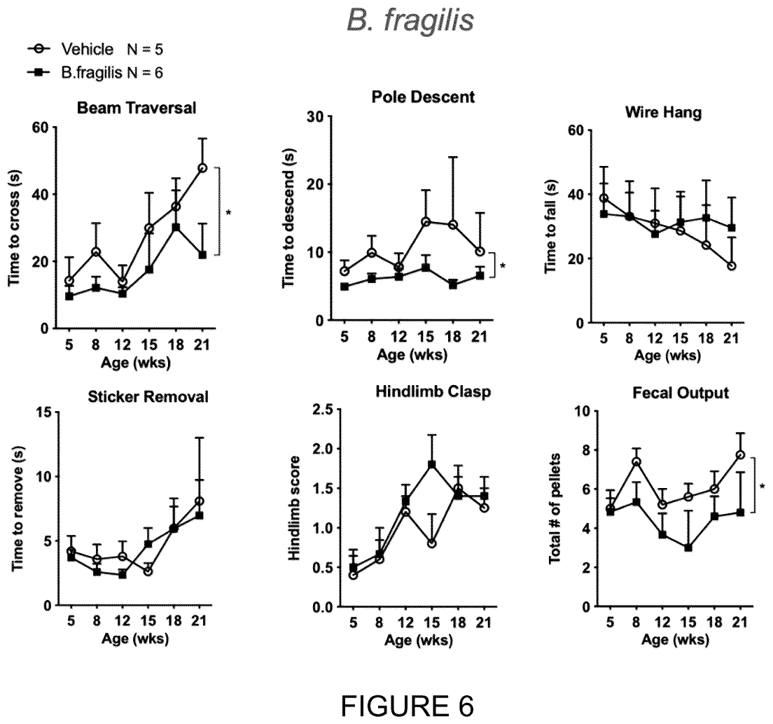

The test compositions that include P. histicola or F. prausnitzii were supplemented with Bacteroides fragilis. The combination of P. histicola or F. prausnitzii and B. fragilis resulted in increased motor function over time, and significantly decreased levels of phosphorylated αSyn in the midbrain, as shown in FIG. 4 and FIG. 5, which is more robust than treatment with B. fragilis alone, as shown in FIG. 6.

### Example 3

The following example demonstrates an embodiment of a method of using the compositions described herein for treating a human subject having Parkinson's disease.

For the use of P. histicola or F. prausnitzii as a therapeutic for PD, a probiotic supplement or medication was formulated using the whole bacteria in its live or killed form, or its components (for example, outer membrane vesicles), as described in Example 1.

For convenient oral administration of the probiotic to human patients, P. histicola or F. prausnitzii (live or killed) or P. histicola or F. prausnitzii components are encapsulated in a stomach acid-resistant capsule (or pill, tablet) for delivery to the intestines. P. histicola or F. prausnitzii (live or killed) or its components are alternatively incorporated into a food such as yogurt, milk, kefir, milk powder, protein powder, ice cream, juice, tea, kombucha, snack bar, cereal, or cookie. Either of these options is easy for a patient to ingest consistently (at least 2 times a week, but up to several times a day) to achieve a therapeutic effect on any PD symptoms, like decreased tremors, improved motor function, or even slowing of disease spread. This bacterial supplement can be taken at any point in the Parkinson's disease progression but may be more effective if taken early on.

Previously, no bacterial supplements have been used or developed to reduce, ameliorate, inhibit, improve, or treat Parkinson's disease and PD symptoms. Therefore, the compositions provided herein, including compositions that include P. histicola and F. prausnitzii are the first commensal bacteria that can be used for the treatment of PD symptoms in humans in the form of a supplement or medication. Since these bacteria are commensal microbes, the supplementation does not produce undesirable side effects and is not addictive or habituating, like is typical in many medications. In some embodiments, with consistent intake, P. histicola and F. prausnitzii will decrease PD severity by ameliorating motor function, and P. histicola intake will decrease α-synuclein inclusions in patients.

### References

The following references are expressly incorporated herein by reference in their entireties for all purposes.
Braak, H., Ruüb, U., Gai, W. P., & Del Tredici, K. (2003). Idiopathic Parkinson's disease: possible routes by which vulnerable neuronal types may be subject to neuroinvasion by an unknown pathogen. Journal of Neural Transmission, 110(5), 517-536.
Chesselet, M. F., Richter, F., Zhu, C., Magen, I., Watson, M. B., & Subramaniam, S. R. (2012). A progressive mouse model of Parkinson's disease: the Thy1-aSyn (“Line 61”) mice. Neurotherapeutics, 9(2), 297-314.
Downes, J., Hooper, S. J., Wilson, M. J., & Wade, W. G. (2008). Prevotella histicola sp. nov., isolated from the human oral cavity. International Journal of Systematic and Evolutionary Microbiology, 58(8), 1788-1791.
Duncan, S.H., (2002). Growth requirements and fermentation products of Fusobacterium prausnitzii, and a proposal to reclassify it as Faecalibacterium prausnitzii gen. nov., comb. nov. International Journal of Systematic and Evolutionary Microbiology 52, 2141-2146.
Fleming, S. M., Salcedo, J., Fernagut, P. O., Rockenstein, E., Masliah, E., Levine, M. S., & Chesselet, M. F. (2004). Early and progressive sensorimotor anomalies in mice overexpressing wild-type human α-synuclein. Journal of Neuroscience, 24(42), 9434-9440.
Keshavarzian, A., Green, S. J., Engen, P. A., Voigt, R. M., Naqib, A., Forsyth, C. B., & Shannon, K. M. (2015). Colonic bacterial composition in Parkinson's disease. Movement Disorders, 30(10), 1351-1360.
Liddle, R. A. (2018). Parkinson's disease from the gut. Brain Research, 1693, 201-206.
Mangalam, A., Shahi, S. K., Luckey, D., Karau, M., Marietta, E., Luo, N., & Rodriguez, M. (2017). Human gut-derived commensal bacteria suppress CNS inflammatory and demyelinating disease. Cell Reports, 20(6), 1269-1277.
Marietta, E. V., Murray, J. A., Luckey, D. H., Jeraldo, P. R., Lamba, A., Patel, R., & Taneja, V. (2016). Suppression of inflammatory arthritis by human gut-derived Prevotella histicola in humanized mice. Arthritis & Rheumatology, 68(12), 2878-2888.
Martin, R., Miguel, S., Benevides, L., Bridonneau, C., Robert, V., & Langella, P., (2017). Functional Characterization of Novel Faecalibacterium prausnitzii Strains Isolated from Healthy Volunteers: A Step Forward in the Use of F. prausnitzii as a Next-Generation Probiotic. Front Microbiol 8, 1226.
Mulak, A., & Bonaz, B. (2015). Brain-gut-microbiota axis in Parkinson's disease. World Journal of Gastroenterology: WJG, 21(37), 10609.
Petrov, V. A., Saltykova, I. V., Zhukova, I. A., Alifirova, V. M., Zhukova, N. G., Dorofeeva, Y. B., & Mironova, Y. S. (2017). Analysis of gut microbiota in patients with Parkinson's disease. Bulletin of Experimental Biology and Medicine, 162(6), 734-737.
Rockenstein, E., Mallory, M., Hashimoto, M., Song, D., Shults, C. W., Lang, I., & Masliah, E. (2002). Differential neuropathological alterations in transgenic mice expressing α-synuclein from the platelet-derived growth factor and Thy-1 promoters. Journal of Neuroscience Research, 68(5), 568-578.
Sampson, T. R., Debelius, J. W., Thron, T., Janssen, S., Shastri, G. G., Ilhan, Z. E., & Chesselet, M. F. (2016). Gut microbiota regulate motor deficits and neuroinflammation in a model of Parkinson's disease. Cell, 167(6), 1469-1480.
Sarrabayrouse, G., Bossard, C., Chauvin, J.-M., Jarry, A., Meurette, & F., Sokol, H., Jotereau, F. (2014). CD4CD8αα Lymphocytes, A Novel Human Regulatory T Cell Subset Induced by Colonic Bacteria and Deficient in Patients with Inflammatory Bowel Disease. PLoS Biol 12, e1001833.
Scheperjans, F., Aho, V., Pereira, P. A., Koskinen, K., Paulin, L., Pekkonen, E., & Kinnunen, E. (2015). Gut microbiota are related to Parkinson's disease and clinical phenotype. Movement Disorders, 30(3), 350-358.
Sokol, H., Pigneur, B., Watterlot, L., Lakhdari, O., Bermúdez-Humarán, & P., Langella, P. (2008). Faecalibacterium prausnitzii is an anti-inflammatory commensal bacterium identified by gut microbiota analysis of Crohn disease patients. PNAS 105, 16731-16736.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods, compositions, kits, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Wherever a method of using a composition (e.g., a composition comprising, consisting essentially of, or consisting of one or more microbial organisms or components thereof) is disclosed herein, the corresponding composition for use is also expressly contemplated. For example, for the disclosure of a method of inhibiting, reducing, delaying, preventing, or ameliorating a neurodegenerative disorder, or one or more symptoms of a neurodegenerative disorder in a subject, comprising administering a composition as provided herein, the corresponding composition for use in inhibiting, reducing, delaying, preventing, or ameliorating a neurodegenerative disorder, or one or more symptoms of a neurodegenerative disorder is also contemplated.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1-46. (canceled)

47. A composition comprising:
one or more isolated microbial organisms or a component of the isolated microbial organism, wherein the one or more isolated microbial organisms comprise Bacteroides, Prevotella, Faecalibacterium, a mixture thereof, or a component derived therefrom,
wherein the composition is formulated as a supplement, a powder, a pill, a tablet, a capsule, a pharmaceutical composition, a nutraceutical composition, or a probiotic composition.

48. The composition of claim 47, wherein the isolated microbial organism is cultured bacteria.

49. The composition of claim 47, wherein the components of the isolated microbial organism comprise bacterial outer membrane vesicles derived from the isolated microbial organism.

50. The composition of claim 47, wherein the composition consists essentially of Prevotella histicola and Faecalibacterium prausnitzii.

51. The composition of claim 47, wherein the composition comprises Prevotella histicola and Faecalibacterium prausnitzii, or a component thereof.

52. The composition of claim 50, wherein the P. histicola and the F. prausnitzii are in a single composition.

53. The composition of claim 50, wherein the Prevotella histicola is present in an amount of at least 10⁸ colony forming units (cfu) and the Faecalibacterium prausnitzii is present in an amount of at least 10⁸ cfu.

54. The composition of claim 51, further comprising Bacteroides fragilis, or a component thereof.

55. The composition of claim 47, wherein the composition is formulated in an acid-resistant formulation.

56. The composition of claim 47, wherein the composition is formulated for controlled release within the lower intestine or colon.

57. The composition of claim 47, further comprising an anti-inflammatory agent.

58. A method of inhibiting, reducing, delaying, preventing, or ameliorating a synucleinopathy, or one or more symptoms of a synucleinopathy, the method comprising:
administering to a subject in need the composition of claim 47.

59. The method of claim 58, wherein the synucleinopathy is a neurodegenerative disorder, an enteric nervous system disorder, or inflammation associated thereto.

60. The method of claim 58, wherein said subject is one that has been identified or selected as being at risk for developing or already having Parkinson's disease, such as by clinical or diagnostic evaluation.

61. The method of claim 58, wherein the subject is selected as in need of the composition if a presence of Prevotella histicola or Faecalibacterium prausnitzii in an intestinal sample is lower than a predetermined level or control.

62. The method of claim 58, wherein the subject in need has an abnormal level of aggregation of α-synuclein (αSyn).

63. The method of claim 58, wherein the method improves one or more gastrointestinal functions of the subject.

64. The method of claim 58, wherein the method reduces αSyn aggregated in the subject.

65. The method of claim 58, wherein the method reduces neuroinflammation in the subject.

66. The method of claim 58, wherein the method increases levels of gut Prevotella histicola or Faecalibacterium prausnilzii.
